(19) United States
(12) Patent Application Publication
(10) Pub. No.: US 2022/0340908 A1
(43) Pub. Date: Oct. 27, 2022

(54) METHODS FOR DEPLETION OF DELETERIOUS MITOCHONDRIAL GENOMES

(71) Applicant: University of Massachusetts, Boston, MA (US)

(72) Inventors: Cole Haynes, Holden, MA (US); Qiyuan Yang, Shrewsbury, MA (US)

(21) Appl. No.: 17/642,986

(22) PCT Filed: Sep. 25, 2020
(86) PCT No.: PCT/US2020/052792

§ 371 Date: Mar. 15, 2022

Related U.S. Application Data
(60) Provisional application No. 62/905,848, filed on Sep. 25, 2019.
(60) Provisional application No. 63/072,006, filed on Aug. 28, 2020.

(51) Int. Cl. C12N 15/113; A61K 38/06; A61K 31/365; A61K 31/36; A61K 31/496; A61K 31/277; A61P 43/00

(52) U.S. Cl. CPC C12N 15/1137; A61K 38/06; A61K 31/365; A61K 31/36; A61K 31/496; A61K 31/277; A61P 43/00; C12N 2310/11; C12N 2310/14; C12N 2310/3513; C12N 2310/3231

(57) ABSTRACT

Methods and compositions to deplete deleterious mitochondrial genomes (ΔmtDNAs), resulting in a compensatory increase in WT mtDNAs, by inhibition of LONP1, e.g., by inhibitory nucleic acids (including RNAi); inducing mutations that prevent the protease from binding mtDNA; or administering an inhibitor, e.g., the clinically relevant compound CDDO-Me (Bardoxolone), all of which result in the preferential loss of ΔmtDNAs.

### CLAIM OF PRIORITY

This application claims the benefit of U.S. Provisional Patent Application Ser. Nos. 62/905,848, filed on Sep. 25, 2019, and 63/072,006, filed on Aug. 28, 2020. The entire contents of the foregoing are hereby incorporated by reference.

### FEDERALLY SPONSORED RESEARCH OR DEVELOPMENT

This invention was made with Government support under Grant Nos. AG040061 and AG047182 awarded by the National Institutes of Health. The Government has certain rights in the invention.

### TECHNICAL FIELD

Described herein are methods and compositions to deplete deleterious mitochondrial genomes (ΔmtDNAs), resulting in a compensatory increase in WT mtDNAs. The methods include inhibition of LONP, e.g., by RNAi, inducing mutations that prevent the protease from binding mtDNA, or administering an inhibitor, e.g., the clinically relevant compound CDDO-Me (Bardoxolone), all of which result in the preferential loss of ΔmtDNAs.

### BACKGROUND

Deleterious mitochondrial genome (ΔmtDNA) accumulation underlies inherited mitochondrial diseases and syndromes (Hahn, A. & Zuryn, S. Trends Cell Biol 29, 227-240 (2019); Pereira, C. V. & Moraes, C. T. Front Biosci (Landmark Ed) 22, 991-1010 (2017); Larsson, N. G & Clayton, D. A. Annu Rev Genet 29, 151-178 (1995); Schon, E. A., et al., Trends Mol Med 16, 268-276 (2010)) and contributes to the aging-associated decline in mitochondrial function in otherwise healthy individuals (Greaves, L. C. et al. PloS Genet 10, e1004620 (2014)). Heteroplasmy occurs when a deleterious mtDNA (ΔmtDNA) clonally expands to reach >50% of the cellular mtDNA population causing oxidative phosphorylation (OXPHOS) dysfunction.

### SUMMARY

As shown herein, the ATP-dependent protease LONP-1 is responsible for the biased interaction between ATFS-1 and ΔmtDNAs in a heteroplasmic C. elegans model. ATFS-1 is imported into mitochondria, where it is degraded (see, e.g., Nargund et al., Science. 2012 Aug. 3; 337(6094):587-90). However, if the organelle is dysfunctional, ATFS-1 fails to be degraded and binds mtDNA. As shown herein, inhibition of LONP-1 causes ATFS-1 to bind WT and ΔmtDNA equally. Thus, LONP-1 is required for the preferential interaction between ATFS-1 and ΔmtDNA. The present data indicates that when ATFS-1 binds mtDNAs, it promotes replication. For mitochondrial dysfunction caused by a deleterious mtDNA, the accumulation of ATFS-1 promotes replication of the ΔmtDNA.

Thus, provided herein are methods for depleting deleterious mitochondrial genomes (ΔmtDNAs) in a cell. The methods include administering an effective amount of an inhibitor of LONP1. Also provided herein are compositions comprising an inhibitor of LONP1, for use in a method for depleting deleterious mitochondrial genomes (ΔmtDNAs) in a cell.

In some embodiments, administering the inhibitor results in a compensatory increase in wild type (WT) mtDNAs.

In some embodiments, the inhibitor of LONP1 is an inhibitory nucleic acid targeting LONP1 or ATF5. In some embodiments, the inhibitory nucleic acid targeting LONP1 or ATF5 is an antisense oligonucleotide, single- or double-stranded RNA interference (RNAi) compound. In some embodiments, the inhibitory nucleic acid targeting LONP1 is or comprises a locked nucleic acid (LNA) or peptide nucleic acid (PNA).

In some embodiments, the inhibitor of LONP1 is a small molecule inhibitor, e.g., an oleanane triterpenoid; MG262 (Z-Leu-Leu-Leu-B(OH)₂); MG132 (carbobenzoxy-Leu-Leu-leucinal); Obtusilactone A (OA); or (-)-sesamin, or trazadone. In some embodiments, the oleanane triterpenoid is 2-cyano-3, 12-dioxooleana-1,9(11)-dien-28-oic acid (CDDO), or a derivative thereof. In some embodiments, the derivative of CDDO is a methyl ester derivative (CDDO-Me) or imidazole derivative (CDDO-Im).

In some embodiments, the cell is in a mammalian subject, preferably a human subject. As one of skill in the art will appreciate, where an inhibitory nucleic acid is used, it is preferably designed to target a LONP1 sequence from the same species as the subject.

In some embodiments, the cell is in a subject who has a disorder associated with ΔmtDNAs. In some embodiments, the disorder is Leigh Syndrome (Subacute necrotizing encephalomyopathy); Kearns-Sayre Syndrome (KSS); Neuropathy, Ataxia and Retinitis Pigmentosa (NARP) Syndrome; Leber Hereditary Optic Neuropathy (LHON); mitochondrial encephalopathy with lactic acidosis and strokelike episodes (MELAS); Chronic Progressive External Ophthalmoplegia (CPEO); Mitochondrial Neuro-GastroIntestinal Encephalopathy (MNGIE); myoclonic epilepsy with ragged-red fibres (MERRF).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-I. Enriched binding of ATFS-1 and POLG to ΔmtDNAs in heteroplasmic worms. (A) ATFS-1/UPR^{mt} signaling schematic. (B) ATFS-1 ChIP-seq profile of the entire mitochondrial genome (mtDNA) of wild-type worms raised on spg-7(RNAi). (C and D) Images of TMRE-stained micrographs (C) and TMRE quantification (D) of wild-type and heteroplasmic(ΔmtDNA) worms raised on control(RNAi) or wild-type worms raised on spg-7(RNAi). Scale bar, 10 μm. (E) Immunoblots of wild-type and ΔmtDNA worms raised on control(RNAi) or wild-type worms raised on spg-7(RNAi) after fractionation into total lysate (T), post-mitochondrial supernatant (S), and mitochondrial pellet (M). Tubulin (Tub) and the OXPHOS component (NDUFS3) are used as loading controls. Arrow is mitochondrial-localized ATFS-1. (F) Workflow of ATFS-1 or POLG IP-mtDNA and quantification of wild-type mtDNA, ΔmtDNA and total mtDNAs in heteroplasmic worms. (G) Quantification of total mtDNA following ATFS-1 IP-mtDNA in wild-type homoplasmic or heteroplasmic worms. (H) Quantification of wild-type mtDNA and ΔmtDNA following ATFS-1 IP-mtDNA in heteroplasmic worms. Post-lysis/Input ΔmtDNA ratio was 59%. (I) Quantification of wild-type mtDNA and ΔmtDNA following POLG IP-mtDNA in heteroplasmic worms. Post-lysis/Input ΔmtDNA ratio was 59%. n=3; error bars mean±S.E.M.; *p<0.05 (Student's t-test).

FIGS. 2A-G Mitochondrial-localized ATFS-1 is sufficient to maintain ΔmtDNAs. (A) ATFS-1^{ΔNLS}/UPR^{mt} signaling schematic. (B) Photomicrographs of wild-type, atfs-1(et18) and atfs-1(et18)^{ΔNLS};hsp-6ₚᵣ::gfp worms. (Scale bar 0.1 mm). (C) Expression level of hsp-6 mRNA in wild-type, atfs-1(et18) or atfs-1(et18)^{ΔNLS} worms examined by qRT-PCR. (D) Immunoblots of lysates from wild-type and ATFS-1^{ΔNLS} worms raised on control(RNAi) or lonp-1(RNAi). ATFS-1 or ATFS-1^{ΔNLS} are indicated with an arrowhead. (E) ΔmtDNA quantification as determined by qPCR in heteroplasmic wild-type, atfs-1^{ΔNLS} and atfs-1(null) worms. (F) Quantification of wild-type mtDNA and ΔmtDNA following ATFS-1 IP-mtDNA in heteroplasmic wild-type or atfs-1^{ΔNLS} worms. Post lysis/Input ΔmtDNA ratio in wild-type worms was 53% in wild-type and 41.8% in atfs-1^{ΔNLS} worms. (G) Quantification of mtDNA following POLG IP-mtDNA in homoplasmic wild-type worms and homoplasmic atfs-1(null) worms raised on control(RNAi) or spg-7(RNAi). n=3; error bars mean±S.E.M.; *p<0.05 (Student's t-test).

FIGS. 3A-H. Mitochondrial-localized ATFS-1-dependent mtDNA replication is negatively regulated by LONP-1. (A) Quantification of mtDNA in wild-type worms following IP-mtDNA using FLAG or control (Mock) antibody in LONP-1^{FLAG} worms. (B) ChIP-seq profile of mtDNA from homoplasmic LONP-1^{FLAG} worms raised on control(RNAi) using FLAG antibody. ATFS-1 ChIP-seq profile from homoplasmic worms raised on spg-7(RNAi) (as in FIG. 1B). (C) ΔmtDNA and wild-type mtDNA quantification following ATFS-1 IP-mtDNA in heteroplasmic worms raised on control(RNAi) or lonp-1(RNAi). (D) ΔmtDNA quantification in heteroplasmic worms raised on control(RNAi) or lonp-1(RNAi). (E) ΔmtDNA and wild-type mtDNA quantification following POLG IP-mtDNA in heteroplasmic worms raised on lonp-1(RNAi). Post-lysis/Input ΔmtDNA ratio was 32.8%. (F) Quantification of total mtDNA following ATFS-1 IP-mtDNA in wild-type or atfs-1(null) worms raised on control(RNAi) or lonp-1(RNAi). (G) Quantification of mtDNA in homoplasmic wild-type and atfs-1(null) worms raised on control(RNAi) or lonp-1(RNAi). (H) Quantification of total mtDNA following POLG IP-mtDNA in homoplasmic wild-type worms raised on control(RNAi) or lonp-1(RNAi). n=3; error bars mean±S.E.M.; *p<0.05 (Student's t-test).

FIGS. 4A-G. LONP1 inhibition reduces ΔmtDNAs and improves OXPHOS function in heteroplasmic human cells. (A and B) Quantification of KSS ΔmtDNA. KSS cells treated with control siRNA or hLONP1 siRNA. (C) Cell viability of WT (143b) and CoxI G6930A cells exposed to various concentrations of CDDO for 72 hours. (D) Percentage of CoxI G6930A mtDNA in cells treated with DMSO, 0.1 μM or 0.25 μM CDDO for 3, 5, 10 or 18.5 weeks, n=3; error bars mean±S.E.M.; *p<0.05 (Student's t-test). (E-G) Oxygen consumption rates (OCR) of CoxI G6930A cells treated with DMSO, 0.1 μM or 0.25 μM CDDO for 3, 5, 10 or 18.5 weeks (E). (F) Quantification of basal respiration. (G) Quantification of respiratory capacity. (n>=10, mean±S.E.M,*p<0.05).

FIGS. 5A-G (A) Quantification of total mtDNA in homoplasmic wild-type worms (N2) and uaDf5 heteroplasmic worm. (B) POLG immunoblot of wild-type worms following fractionation into total lysate (T), post-mitochondrial supernatant (S), and mitochondrial pellet (M). Tubulin (Tub) and the OXPHOS protein (NDUFS3) serve as loading controls. (C) POLG immunoblot of lysates from wild-type worms raised on control or polg(RNAi). Tubulin (Tub) serves as a loading control. (D) mtDNA quantification following IP-mtDNA using POLG or non-specific (Mock) antibodies in wild-type worms. (E) HMG-5/TFAM Immunoblots of lysates from wild-type worms raised on control or hmg-5/tfam(RNAi). Tubulin (Tub) serves as a loading control. (F) HMG-5/TFAM immunoblot of wild-type worms following fractionation into total lysate (T), post-mitochondrial supernatant (S), and mitochondrial pellet (M). Tubulin (Tub) and the OXPHOS component (NDUFS3) are loading controls. (G) Quantification of wild-type mtDNA and ΔmtDNA following TFAM IP-mtDNA in heteroplasmic worms. Post-lysis/Input ΔmtDNA ratio was 62.7%. n=3; error bars mean±S.E.M.; *p<0.05 (Student's t-test).

FIGS. 6A-C. (A) ATFS-1 schematic highlighting the R (Arg) to A (Ala) amino acid substitution to impair the nuclear localization sequence (NLS) within ATFS-1 yielding ATFS-1^{ΔNLS}; shown are wild type (SEQ ID NO:33) and ATFS-1^{ΔNLS} (SEQ ID NO:34). (B) Expression level of hsp-6 mRNA in wild-type and atfs-1^{ΔNLS} worms raised on control(RNAi) or spg-7(RNAi) examined by qRT-PCR. (C) Quantification of total mtDNA following POLG IP-mtDNA in atfs-1^{ΔNLS} and atfs-1(null) worms raised on control(RNAi) or spg-7(RNAi). n=3; error bars mean±S.E.M.; *p<0.05 (Student's t-test).

FIGS. 7A-E. (A) LONP-1 immunoblots of lysates from wild-type worms raised on control(RNAi) or lonp-1(RNAi). Tubulin (Tub) serves as a loading control. (B) FLAG Immunoblots of LONP-1^{FLAG} worms following fractionation into total lysate (T), post-mitochondrial supernatant (S), and mitochondrial pellet (M). Tubulin (Tub) and the OXPHOS component NDUFS3 are loading controls. (C) FLAG immunoblots of wild-type and LONP-1^{FLAG} worms demonstrating expression of epitope-tagged LONP-1. Tubulin (Tub) serves as a loading control. (D) Images of wild-type or LONP-1^{FLAG} worms 48 hours after synchronization indicating worms expressing LONP-1^{FLAG} at the endogenous locus develop normally (Scale bar 1 mm). (E) Fluorescent photomicrographs of wild-type hsp-6ₚᵣ::gfp or lonp-1^{FLAG};hsp-6ₚᵣ::gfp worms 48 hours after synchronization indicating worms expressing LONP-1^{FLAG} do not cause UPR^{mt} activation. (Scale bar 0.05 mm).

FIGS. 8A-D. (A) IP-mtDNA using ATFS-1 or LONP-1 antibodies in heteroplasmic worms followed by quantification of total mtDNA (both wild-type and ΔmtDNA) indicating that LONP-1 binds 15-fold more mtDNA than ATFS-1. (B) Quantification of wild-type mtDNA and ΔmtDNA following LONP-1 IP-mtDNA in heteroplasmic worms. Post-lysis/Input ΔmtDNA ratio was 54%. The results indicate LONP-1 binding is not enriched at either wild-type mtDNA or ΔmtDNA, unlike ATFS-1 (FIG. 1H). (C) LONP-1 consensus binding site within mtDNA (SEQ ID NO:35). (D) Schematic of the putative ATFS-1 (SEQ ID NO:36) and LONP-1 (SEQ ID NO:37) binding sites within the mtDNA non-coding region (NCR) highlighting the proximity of both sites (˜200 base pairs). n=3; error bars mean±S.E.M.; *p<0.05 (Student's t-test).

FIGS. 9A-C. (A) ΔmtDNA quantification in atfs-1^{ΔNLS} heteroplasmic worms raised on control(RNAi) or lonp-1(RNAi). (B) ΔmtDNA and wild-type mtDNA quantification following TFAM IP-mtDNA in heteroplasmic worms raised on lonp-1(RNAi). Post-lysis/Input ΔmtDNA ratio was 34%. (C) Total mtDNA quantification in wild-type homoplasmic and atfs-1^{ΔNLS} homoplasmic worms raised on control (RNAi) or lonp-1 (RNAi). n=3; error bars mean±S.E.M.; *p<0.05 (Student's t-test).

FIGS. 10A-E. (A) LONP1 immunoblots from KSS heteroplasmic cells treated with hLONP1 or NC (control) siRNA. Tubulin (Tub) serves as a loading control (B) Cell viability of WT (143b) and KSS ΔmtDNA cells exposed to various concentrations of CDDO for 72 hours. (C) Quantification of G6930A mtDNA percentage following treatment with DMSO, 0.1 μM CDDO, or 0.25 μM CDDO at the indicated time points up to 130 days. n=3; error bars mean±S.E.M.; *p<0.05 (Student's t-test). (D) ΔmtDNA quantification in KSS heteroplasmic cells treated with DMSO, 0.1 μM CDDO, or 0.25 μM for 4 or 13 weeks. n=3; error bars mean±S.E.M. (E) Basal respiration of KSS heteroplasmic cells treated with DMSO (ctrl), 0.1 μM or 0.25 μM CDDO for 4 or 13 weeks (n=14-16, mean±S.E.M,*p<0.05).

FIGS. 11A-E. (A) ATF5 immunoblots of lysates from control or KSS cells treated with ATF5 shRNAs. (B) Expression level of ATF5 mRNA in control or ATF5 shRNA-treated KSS cells examined by qRT-PCR. (C) ΔmtDNA quantification in control or ATF5-treated shRNA KSS cells. (D) Quantification of the percentage of ΔmtDNA percentage in control or ATF5 shRNA-treated KSS cells as determined by qPCR. (E) ΔmtDNA quantification in control or Trazodone-treated KSS cells. n=3; error bars mean±S.E.M.; *p<0.05 (Student's t-test).

### DETAILED DESCRIPTION

Mitochondrial genomes (mtDNA) encode essential genes including components for oxidative phosphorylation (OXPHOS), which is responsible for the generation of most cellular energy in the form of ATP. Each mitochondrion harbors multiple mtDNAs and most cells harbor hundreds of mtDNAs. Heteroplasmy occurs when a deleterious mutant mtDNA clone expands to a high enough percentage relative to wild-type mtDNAs to perturb OXPHOS function. Interestingly, expression of mitochondrial-targeted nucleases that specifically cleave deleterious mtDNAs suggest that a modest reduction in the ratio of mutant to wild-type mtDNA is sufficient to improve mitochondrial function (Srivastava and Moraes, Human molecular genetics 10, 3093-3099 (2001); Tanaka et al., Journal of biomedical science 9, 534-541 (2002)).

Over 270 different mitochondrial genome (mtDNA) mutations have been found to cause disease (Pereira, C. V. & Moraes, C. T. Front Biosci (Landmark Ed) 22, 991-1010 (2017)), which occur in 1:4000 individuals (Chinnery, P. F. et al. Ann Neurol 48, 188-193 (2000)). Inheritance of mutant mtDNAs is not Mendelian as the mitochondrial genome is strictly transmitted through the maternal germ line. Thus, one must inherit a relatively high percentage of mutant mtDNAs relative to wildtype mtDNAs. Because there are 100s-1000s mtDNAs, pathology only occurs when the mutant (A) mtDNA reaches ˜60-70% of the total mtDNA pool. Thus, even reducing the percentage of ΔmtDNAs relatively modestly (e.g., by about 10%) can provide impressive therapeutic effects. The inheritance of a high percentage of mutant mtDNAs that result in mitochondrial diseases or syndromes occurs in 1:5000 births (e.g., Leigh Syndrome, NARP Syndrome, MELAS, LHON, MNGIE, MERRF, KSS, CPEO). Symptoms are typically loss of neuromuscular function but also including loss of vision, deafness, diabetes, stroke, seizures and cardiac arrhythmia. (see, e.g., Clinical Mitochondrial Medicine, Chinnery and Keogh 2018).

In addition to inheriting a high percentage of ΔmtDNAs, at some level everyone incurs ΔmtDNAs, most likely due to errors in replication. As somatic cells such as muscles and neurons age, the ΔmtDNAs are often preferentially propagated at the expense of wildtype mtDNAs leading to loss of OXPHOS in individual cell types, which likely impacts muscle and neuronal tissues. Mitophagy has been shown to limit the accumulation of ΔmtDNAs by specifically degrading dysfunctional mitochondria, which likely harbor ΔmtDNAs (Suen et al., Proceedings of the National Academy of Sciences of the United States of America 107, 11835-11840 (2010)). ΔmtDNA propagation is also suppressed by a mechanism that limits protein synthesis on the outer membrane of dysfunctional mitochondria (Zhang et al., Molecular cell 73, 1127-1137. e1125 (2019)). Importantly, ΔmtDNAs accumulation is exacerbated in the affected tissues of Alzheimer's and Parkinson's Disease patients, suggesting they may impact neuronal function and pathology.

While the initial mtDNA mutation or deletion is likely formed by aberrant mtDNA replication (Larsson, Annu Rev Biochem 79, 683-706 (2010)), the mechanisms that propagate deleterious mtDNAs and maintain them at a percentage that causes OXPHOS defects in mitochondrial diseases (Hahn & Zuryn, Trends Cell Biol 29, 227-240 (2019); Pereira & Moraes, Front Biosci (Landmark Ed) 22, 991-1010 (2017); Larsson & Clayton, Annu Rev Genet 29, 151-178 (1995); Schon et al., Trends Mol Med 16, 268-276 (2010)), aging (Greaves et al., PloS Genet 10, e1004620 (2014)) as well as Parkinson's Disease (Bender et al., Nat Genet 38, 515-517 (2006)) remain unclear. Depending on the severity of the mtDNA lesion, the ΔmtDNA percentage must reach a specific threshold to cause disease suggesting that a modest reduction in the ratio of mutant to wild-type mtDNA may significantly improve mitochondrial function.

The transcription factor ATFS-1 mediates the mitochondrial unfolded protein response (UPR^{mt}) (Nargund et al., Science 337, 587-590 (2012); Nargund et al., Mol Cell 58, 123-133 (2015)) and is required to maintain ΔmtDNAs in heteroplasmic C. elegans strains (Lin et al., Nature 533, 416-419 (2016); Gitschlag et al., Cell Metab 24, 91-103 (2016); Tsang & Lemire, Biochem Cell Biol 80, 645-654 (2002)). As shown herein, mitochondrial-localized ATFS-1 promotes mtDNA replication in dysfunctional mitochondrial compartments, leading to biased replication of ΔmtDNAs. Strikingly, ATFS-1 preferentially interacted with ΔmtDNAs to promote mtDNA polymerase gamma (POLG-1) binding and replication. The ATFS-1-ΔmtDNA bias required the ATP-dependent, mitochondrial protease LONP-1 (Bota & Davies, Nat Cell Biol 4, 674-680 (2002)), which degrades ATFS-1 in the mitochondrial matrix of functional compartments (Nargund et al., Science 337, 587-590 (2012); Nargund et al., Mol Cell 58, 123-133 (2015)). (Note that the mammalian homologue of the worm gene LONP-1 is referred to herein as LONP or LONP1).

It has been postulated for over 20 years that shifting of the wild-type:pathogenic mtDNA ratio, either by enhancing replication of wild-type mtDNA or depleting the pathogenic mtDNA could be therapeutic (Taivassalo et al., Hum Mol Genet 8, 1047-1052 (1999)). Considerable work has focused on depleting mutant mtDNAs by targeting nucleases to mitochondria that specifically degrade the pathogenic mtDNAs (Bacman et al., Gene Ther 17, 713-720 (2010); Bacman et al., Nat Med 19, 1111-1113 (2013); Gammage et al., Nat Med 24, 1691-1695 (2018); Reddy et al., Cell 161, 459-469 (2015)). Despite the promise of these approaches, considerable challenges exist that may limit therapeutic development such as the introduction and delivery of such nucleases to affected tissues. We have discovered a mechanism underlying the preferential replication of pathogenic mtDNAs. This research has revealed 1) strategies to prevent pathogenic mtDNA replication and 2) a therapeutically viable approach to specifically promote replication of healthy mtDNAs and recover mitochondrial function

Genetic and pharmacological inhibition of LONP-1 in C. elegans and human cybrid cells (King & Attardi, Science 246, 500-503 (1989)) prevented the biased ΔmtDNA replication resulting in loss of the ΔmtDNAs. The present findings suggest an evolutionary conserved mechanism where mtDNA-bound LONP-1 (Chen et al., Nucleic Acids Res 36, 1273-1287 (2008)) serves as an internal sensor of organelle function that promotes mtDNA replication in dysfunctional compartments.

Without wishing to be bound by theory, it is believed that when LONP-1 activity declines, ATFS-1 avoids degradation and binds mtDNA to promote replication in an effort to recover the dysfunctional compartment that inadvertently biases ΔmtDNA replication in heteroplasmic cells.

The present results indicate an unanticipated role for mitochondrial-localized ATFS-1 in maintaining deleterious mtDNAs, and suggest the mtDNA-bound protease LONP-1 establishes the biased interaction between ATFS-1 and ΔmtDNAs resulting in preferential replication. Without wishing to be bound by theory, it is believed that this mechanism may have evolved to allow compartment-autonomous regulation of mtDNA replication responsive to functional heterogeneity within the mitochondrial network that may occur during cells growth, which is exacerbated in heteroplasmic cells. LONP-1 proteolytic activity provides a regulatory mechanism to coordinate mtDNA replication with expansion of the mitochondrial network during cell growth or recovery from mitochondrial dysfunction. If a compartment becomes dysfunctional, LONP-1 activity may decline, allowing ATFS-1 and subsequently POLG-1 to bind mtDNA and promote replication. However, if the compartmental dysfunction is caused by localized enrichment of ΔmtDNAs, they are inadvertently replicated, which is impaired by LONP-1 inhibition. Thus provided herein are methods and compositions for depleting ΔmtDNAs, which results in a compensatory increase in WT mtDNAs and improving the WT:ΔmtDNA ratio and mitochondrial function. Inhibition of LONP1, e.g., by inhibitory nucleic acids including RNAi; by introducing mutations that prevent the protease from binding mtDNA; or by inhibitors, e.g., small molecule inhibitors, including the clinically relevant compound CDDO-Me (Bardoxolone; Chin et al., Am J Nephrol 47, 40-47 (2018)), results in the preferential loss of ΔmtDNAs, improves the wild-type:pathogenic mtDNA ratio, recovers mitochondrial function, and leads to a reduction in the associated pathology or risk of pathology.

Methods of Use

The methods described herein include methods for reducing ΔmtDNAs in a cell, e.g., for the treatment of disorders associated with ΔmtDNAs. In some embodiments, the disorder is Leigh Syndrome (Subacute necrotizing encephalomyopathy); Kearns-Sayre Syndrome (KSS); Neuropathy, Ataxia and Retinitis Pigmentosa (NARP) Syndrome; Leber Hereditary Optic Neuropathy (LHON); mitochondrial encephalopathy with lactic acidosis and strokelike episodes (MELAS); Chronic Progressive External Ophthalmoplegia (CPEO); Mitochondrial Neuro-GastroIntestinal Encephalopathy (MNGIE); myoclonic epilepsy with ragged-red fibres (MERRF), some of which are associated with deafness and/or inherited type 2 diabetes. Generally, the methods include administering a therapeutically effective amount of a LONP1 inhibitor as described herein, to a subject who is in need of, or who has been determined to be in need of, such treatment. The methods can include a step of identifying and/or selecting a subject who has a disorder associated with ΔmtDNAs, e.g., identifying and/or selecting them on the basis that they have the disorder. Methods for diagnosing a subject with a disorder associated with ΔmtDNAs are known in the art. A disease may be associated with ΔmtDNAs when: a common disease has atypical features; three or more organ systems are involved (or 1-2 of symptoms listed in table 2 above); or recurrent setbacks/flare ups occur in a chronic disease occur with infections. In some embodiments, a diagnosis is made based in part on the Nijmegen Clinical Criteria for Mitochondrial Disease (see, e.g., Wolf and Smeitink, Neurology. 59(9): 1402-5 (2002); Crawley, “Mitochondrial Disease: Information Booklet for Medical Practitioners,” May 2014, available at amdf.org.au/wp-content/uploads/2014/05/Mito-Medical-Info-Booklet-201405-web.pdf). A definitive diagnosis can include genetic testing, e.g., to identify and confirm the presence of pathogenic mtDNAs, e.g., mutations. For example, a tissue biopsy can be used; e.g., muscle biopsy to obtain morphological, biochemical, and molecular data. Various methods, including sequencing arrays, southern blotting, and/or next generation sequencing (NGS) approaches can be used to identify mutations and optionally determine the percentage or ratio of ΔmtDNA to wt-mtDNA. Whole genome sequencing that includes both the nuclear genome and mitochondrial genome can be used to determine whether the disease is due to a mutation in a nuclear or mitochondrial gene. Following diagnosis, deep sequencing of mtDNA can be used to determine shifts in mutant/wt mtDNA ratios.

In some embodiments, the subject does not have (or has not been diagnosed with) cancer, and/or does not have (or has not been diagnosed with) heart failure unrelated to a mitochondrial disease.

As used in this context, to “treat” means to ameliorate at least one symptom of the disorder associated with associated with ΔmtDNAs. An excess amount of ΔmtDNAs results in tissue specific or systemic deficits; thus, a treatment can result in a reduction in levels of ΔmtDNAs (e.g., relative to wild type mtDNA) and/or an increase in wt-mtDNA, and a return or approach to normal function, e.g., a lessening of symptoms associated with the tissue specific or systemic deficits. Administration of a therapeutically effective amount of a compound described herein for the treatment of a condition associated with associated with ΔmtDNAs will result in an improvement in one or more symptoms of the condition. For example, symptoms can vary depending on the affected tissue(s) and can include one or more of seizures; attention/concentration deficits; headache; stroke; loss of motor control; muscle weakness; pain; fatigue; cardiomyopathy; impaired hearing; impaired liver function; impaired gastric and/or intestinal motility; slowing or stunting of growth; retinitis; diabetes; and optic atrophy. Table 1 below, reproduced from Crawley, “Mitochondrial Disease: Information Booklet for Medical Practitioners,” May 2014, available at amdf.org.au/wp-content/uploads/2014/05/Mito-Medical-Info-Booklet-201405-web.pdf. provides a list of tissues and symptoms.

Without wishing to be bound by theory, the treatment will decrease ΔmtDNAs and/or increase wt-mtDNAs, e.g., resulting in an increased ratio of wt:A mtDNAs, in at least some relevant cells in a tissue of the subject.

An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

CDDO/Bardoxolone doses of 20 mg for 56 consecutive days were well tolerated and yielded positive results in a phase III of chronic kidney disease patients with type 2 diabetes (see Rizk et al., Cardiorenal Med. 2019; 9(5):316-325.). Trazodone doses of 50 mg 300 mg daily are prescribed for depression.

Animal models of relevant conditions can be created, e.g., using restriction endonucleases, e.g., as described in Pinto and Moraes, Biochim Biophys Acta. 2014 August; 1842(8):1198-207, or mitochondria targeted nucleases, e.g., as described in Bacman et al., Methods Enzymol. 2014; 547: 373-397.

TABLE 1

| Organ/System | Possible Problems |
|---|---|
| Brain | Developmental delays, mental retardation/regression, |
| | dementia, seizures (especially atypical or refractory), |
| | coma, neuro-psychiatric disturbances, atypical |
| | cerebral palsy, myoclonus, movement disorders, |
| | ataxia, migraines, strokes. |
| Nerves | Weakness (which may be intermittent), neuropathies, |
| | absent reflexes, fainting, absent or excessive |
| | sweating resulting in temperature regulation |
| | problems. |
| Muscles | Weakness, hypotonia, cramping, muscle pain, |
| | recurrent rhabdomyolysis. |
| Kidneys | Proximal renal tubular wasting resulting in loss of |
| | protein, magnesium, phosphorous, calcium and other |
| | electrolytes, aminoaciduria, nephrotic syndrome. |
| Heart | Conduction defects (e.g., heart blocks, WPW), |
| | cardiomyopathy. |
| Liver | Hypoglycaemia, unexplained liver failure, Valproate- |
| | induced liver failure. |
| Eyes | Visual loss/blindness, optic atrophy, disorders of |
| | extra-ocular muscles, ptosis, retinal degeneration |
| | with signs of night blindness, colour-vision deficits, |
| | pigmentary retinal changes such as retinitis |
| | pigmentosa or ‘salt and pepper’ retinopathy. |
| Ears | Hearing loss and deafness (especially sensorineural). |
| Pancreas | Diabetes and exocrine pancreatic failure (inability to |
| | make digestive enzymes). |
| Systemic | Exercise intolerance not in proportion to weakness, |
| | fatigue, short statue, respiratory problems including |
| | intermittent air hunger, hypersensitive to general |
| | anaesthetics. |
| GIT | Gastro-oesophageal reflux, delayed gastric emptying, |
| | constipation, pseudo-obstruction, chronic or recurrent |
| | vomiting |
| Childhood | IUGR, unexplained hypotonia, weakness, failure to |
| | thrive, or a metabolic acidosis (particularly lactic |
| | acidosis), infantile spasms, microcephaly, ‘SIDS’. |
| Skin | Symmetrical lipomatosis. |
| Endocrine | Diabetes, short stature, hypothyroidism, |
| | hypoparathyroidism. |
| Haematological | Sideroblastic anaemia |

Table 2, reproduced from Crawley, “Mitochondrial Disease: Information Booklet for Medical Practitioners,” May 2014, available at amdf.org.au/wp-content/uploads/2014/05/Mito-Medical-Info-Booklet-201405-web.pdf, provides a list of symptoms that could suggest that a condition is associated with ΔmtDNAs.

TABLE 2

| Clinical | |
|---|---|
| Manifestation | Features suspicious of a mitochondrial disease |
| Sensorineural | Asymmetrical onset |
| hearing loss | Young age of onset |
| | History of partial recovery after an insult, i.e., |
| | reversible |
| | High frequencies affected first |
| Focal neurological | Young age of onset |
| deficits | Preceded by clinical prodrome |
| | Nonvascular territory on neuroimaging |
| | Predominantly grey matter affected |
| | Associated basal ganglia calcification |
| | Good clinical recovery from an ‘event’ |
| | Neuroradiological changes out of proportion to |
| | clinical deficit |
| | Associated focal seizures or status epilepticus |
| | Raised CSF lactate |
| Seizures | Sudden onset status epilepticus |
| | Recurrent physiological trigger |
| | Severe episodes of seizures with good interval |
| | periods (requiring no ACDs for control) |
| | Worsened by sodium valproate |
| Ptosis | Asymmetrical onset |
| | Slowly progressive with little diurnal variation |
| | Accompanying PEO or retinal pigmentary changes |
| Retinal pigmentary | Perimacular distribution |
| changes | No drusen |
| | Non-vision threatening |
| Diabetes | No associated diabetic retinopathy/peripheral |
| | neuropathy with respect to the length of |
| | diabetes onset |
| | Easily controlled with OHA with respect to duration |
| | of diabetes |

Pharmaceutical Compositions and Methods of Administration

The methods described herein include the use of pharmaceutical compositions comprising LONP1 inhibitors as an active ingredient. The compositions are also provided herein.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., oral coenzyme supplementation for subjects with conditions caused by defects in Coenzyme Q10 biosynthesis; thymidine phosphorylase (TP) for subjects with mitochondrial neuro-gastrointestinal encephalomyopathy (MNGIE); and riboflavin for adults with riboflavin transporter disorders. Although some disorders, including MNGIE are caused by mutations in genes including nuclear genes that lead to a reduction in the mtDNA copy number and consequent mitochondrial dysfunction in the affected tissues, the mutation makes it difficult to synthesize mtDNA, so in some embodiments LONP1 inhibitors can be used to increase mtDNA synthesis in these cells. Other supplementary compounds can include vitamin E, thiamine, nicotinamide, creatine, ascorbic acid, vitamin K3, dichloroacetate, alpha ilpoic acid, succinate, biotin, L-carnitine, magnesium ortate, and/or L-arginine.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration. In the present methods, the composition is preferably administered systemically or to the affected tissue.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Small Molecule LONP1 Inhibitors

LONP1 inhibitors include oleanane triterpenoids, e.g., 2-cyano-3, 12-dioxooleana-1,9(11)-dien-28-oic acid (CDDO), or its derivatives, e.g., C-28 methyl ester derivative (CDDO-Me) or imidazole derivative (CDDO-Im) (see Bernstein et al., Blood 119(14):3321-9 (2012)). MG262 (Z-Leu-Leu-Leu-B(OH)₂, a boronic peptide acid) and MG132 (carbobenzoxy-Leu-Leu-leucinal, as a peptide aldehyde) are potent inhibitors of LONP1 protease (Frase et al., Biochemistry. 2006; 45(27):8264-8274; Granot et al., Mol Endocrinol. 2007; 21(9):2164-2177; available from ApexBio). Obtusilactone A (OA) and (-)-sesamin from Cinnamomum kotoense are also inhibitors of LONP1 protease (Wang et al., Cancer Science 101(12):2612-20 (2010)).

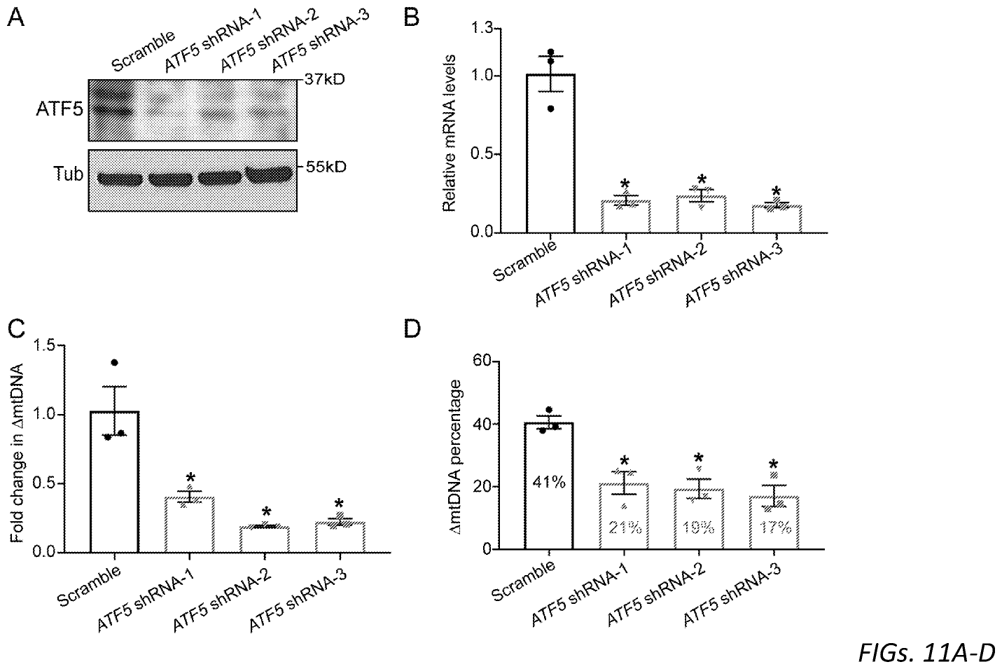

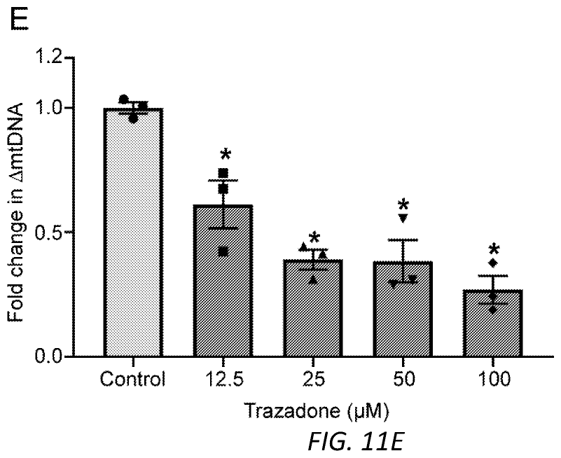

Like CDDO, trazadone also improved heteroplasmy in human cells (see FIGS. 11A-E). The drug is an anti-depressant, but was found to inhibit a stress response pathway (Integrated Stress Response or ISR) that regulates expression of ATF5 and LONP1. (Halliday et al., Brain. 2017 Jun. 1; 140(6):1768-1783).

Inhibitory Nucleic Acids

Inhibitory nucleic acids useful in the present methods and compositions include antisense oligonucleotides, ribozymes, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target LONP1 or ATF5 nucleic acid and modulate (decrease or inhibit) its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112.

Sequences for human LONP1 are known in the art. Exemplary sequences for human LONP1 are available in GenBank at the accession numbers below:

| Nucleic Acid | Protein | Description |
|---|---|---|
| NM_004793.4 | NP_004784.2 | lon protease homolog, |
| | | mitochondrial isoform 1 |
| | | precursor |
| NM_001276479.2 | NP_001263408.1 | lon protease homolog, |
| | | mitochondrial isoform 2 |
| NM_001276480.1 | NP_001263409.1 | lon protease homolog, |
| | | mitochondrial isoform 3 |

Variant 1 encodes the longest isoform 1. Variant 2 is alternatively spliced at the 5′ end compared to variant 1. It uses the same translation start codon as variant 1, however, the encoded isoform 2 lacks a 64 aa protein segment in the 5′ coding region compared to isoform 1. Variant 3 contains an alternate 5′ terminal exon and uses an in-frame downstream start codon compared to variant 1. The encoded isoform 3 has a shorter N-terminus compared to isoform 1.

The genomic sequence is available in GenBank at NC_000019.10, Range 5691834-5720452, complement (Reference GRCh38.p13 Primary Assembly)

Sequences for human ATF5 are known in the art. Exemplary sequences for human LONP1 are available in GenBank at the accession numbers below:

| Nucleic Acid | Protein | Description/Notes |
|---|---|---|
| NM_012068.5 | NP_036200.2 | cyclic AMP-dependent |
| | | transcription factor ATF-5, |
| | | transcript variant 1 |
| NM_001193646.1 | NP_001180575.1 | cyclic AMP-dependent |
| | | transcription factor ATF-5, |
| | | transcript variant 2 |
| | | (also known as alpha) |
| NM_001290746.1 | NP_001277675.1 | cyclic AMP-dependent |
| | | transcription factor ATF-5, |
| | | transcript variant 3 |
| | | (also known as beta) |

Variant (2, also known as alpha) and variant (3, also known as beta) have an alternate 5′ UTR exon, compared to variant 1. Variants 1, 2 and 3 encode the same protein.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%/. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides that can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning. A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

siRNA/shRNA

In some embodiments, the nucleic acid sequence that is complementary to a target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the Rnase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc NatlAcadSci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261:1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce Rnase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; Fluiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Orom et al., Gene. 2006 May 10; 372:137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NHO—CH₂, CH, ˜N(CH₃—O—CH₂ (known as a methylene(methylimino) or MMI backbone], CH₂—O—N(CH₃)—CH₂, CH₂—N(CH₃)—N (CH₃)—CH₂ and O—N(CH₃)—CH₂—CH₂ backbones, wherein the native phosphodiester backbone is represented as O—P—O—CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH2)n CH₃ where n is from 1 to about 10; Ci to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO2; NO2; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the ergerineamics properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, HeIv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or ergerine acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the ergerineamics properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the ergerineamics properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or ergerine acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxgygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%/. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants). Viral vectors that can be used in the present methods and compositions include recombinant retroviruses, adenovirus, adeno-associated virus, alphavirus, and lentivirus.

A preferred viral vector system useful for delivery of nucleic acids in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. For example, AAV9 has been shown to efficiently cross the blood-brain barrier. Moreover, the AAV capsid can be genetically engineered to increase transduction efficient and selectivity, e.g., biotinylated AAV vectors, directed molecular evolution, self-complementary AAV genomes and so on. In some embodiments, AAV9 is used.

Alternatively, retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986). In some embodiments, Ad5 is used.

Alphaviruses can also be used. Alphaviruses are enveloped single stranded RNA viruses that have a broad host range, and when used in gene therapy protocols alphaviruses can provide high-level transient gene expression. Exemplary alphaviruses include the Semliki Forest virus (SFV), Sindbis virus (SIN) and Venezuelan Equine Encephalitis (VEE) virus, all of which have been genetically engineered to provide efficient replication-deficient and -competent expression vectors. Alphaviruses exhibit significant neurotropism, and so are useful for CNS-related diseases. See, e.g., Lundstrom, Viruses. 2009 June; 1(1): 13-25; Lundstrom, Viruses. 2014 June; 6(6): 2392-2415; Lundstrom, Curr Gene Ther. 2001 May; 1(1):19-29; Rayner et al., Rev Med Virol. 2002 September-October; 12(5):279-96.

In general, the vector can be engineered to include a mitochondrial localization signals (MLSs) or mitochondrial targeting sequence (MTS), e.g., COX8A N-terminal MLS, yeast CoxIV N-terminal MLS

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-0 atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

Mitochondria Targeted Nucleases

Alterations in LONP1 expression levels or activity can also be achieved by using mitochondria targeted nucleases, e.g., Zinc Fingers or TALENS, that target the LONP1 sequence. In some embodiments, the methods include inducing mutations in basic amino acids (arginine/lysines) between residues 458 and 500 (numbered with reference to NP_004784) to reduce LONP1 binding to mtDNA; for example, mutating lysine 472 and arginine 482 to glutamic acids. Switching from basic amino acids to acidic residues will impair binding to mtDNA similar to the homologous amino acids and mutations in C. elegans Lonp-1.

Such nucleases can be delivered, e.g., using a viral vector as described herein or known in the art, e.g., including a nuclear export sequences, e.g., murine minute virus NS2 sequence. Nucleases with obligate hetero-dimeric FokI domains are preferably used to increase specificity. See, e.g., Bacman et al., Methods Enzymol. 2014; 547: 373-397; Gammage et al., Trends Genet. 2018 February; 34(2): 101-110; Rai et al., Essays Biochem. 2018 Jul. 20; 62(3):455-465, and references described therein. CRISPR/Cas9 has also been reported to work in mitochondria, see, e.g., Jo et al., Biomed Res Int. 2015; 2015:305716.

### EXAMPLES

The invention is further described in the following examples, which do not limit the scope of the invention described in the claims.

Methods

The following materials and methods were used in the Example set forth below, unless otherwise indicated.

Worm Strains

The reporter strain hsp-6ₚᵣ::gfp for visualizing UPR^{mt} activation was previously described (1). N2 (wild-type), and ΔmtDNA (or uaDf5) were obtained from the Caenorhabditis Genetics Center (Minneapolis, Minn.). The atfs-1(et18) strain was a gift from Mark Pilon. The atfs-1(null), or atfs-1(cmh15), strain was generated via CRISPR-Cas9 in wild-type worms as previously described (22). The crRNAs (Integrated DNA Technologies) were co-injected with purified Cas9 protein, tracrRNA (Integrated DNA Technologies), and the dpy-10 co-injection marker as described (34). atfs-1-1^{ΔNLS} was introduced into both wild-type worms and the hsp-6ₚᵣ::gfp reporter strain via CRISPR-Cas9 (crRNAs and replacement sequence listed in Table A). lonp-1^{FLAG} was introduced into both wild-type worms and the hsp-6ₚᵣ::gf reporter strain via CRISPR-Cas9. Each strain was outcrossed at least 5 times. Unless otherwise noted, all worms were harvested between the late L3 and early L4 stages.

C. elegans ΔmtDNA and Human Cybrid Cell KSS mtDNA Quantification

The described worms were synchronized by bleaching and raised on lonp-1(RNAi) seeded plates for 3 days until they reached adulthood. The worms were synchronized again and seeded on lonp-1(RNAi) and harvested once they reached the L4 stage. All qPCR results are presented as technical replicates, but each experiment has been repeated three or more times. mtDNA quantification was determined by qPCR as described (11). Wild-type mtDNA and ΔmtDNA quantification was performed using qPCR-based methods similar to previously described assays (11). 50-60 worms were harvested in 35 μl of lysis buffer (50 mM KCl, 10 mM Tris-HCl (pH 8.3), 2.5 mM MgCl2, 0.45% NP-40, 0.45% Tween 20, 0.01% gelatin, with freshly added 200 μg/ml proteinase K) and frozen at −80° C. for 20 min prior to lysis at 65° C. for 80 min. Relative quantification was used for determining the fold changes in mtDNA between samples. 1 μl of lysate was used in each triplicate qPCR reaction. qPCR was performed using the iQ™ SYBR® Green Supermix and the Biorad qPCR CFX96™ (Bio-Rad Laboratories). Primers that specifically amplify wild-type or ΔmtDNA are listed in Table A, as are primers that amplify both wild-type and ΔmtDNA (Total mtDNAs). Primers that amplify a non-coding region near the nuclear-encoded ges-1 gene were used as an internal control for normalization (Table A).

For human patient fibroblast cell lines, wild-type and ΔKSS primers were used to detect wild-type mtDNA or ΔKSS mtDNA. Primers that amplify a sequence within the B2M (Human 02 myoglobin) gene were used as an internal control for normalization. Absolute quantification was also performed to determine the percentage or ratio of KSS ΔmtDNA relative to total mtDNA (KSS ΔmtDNA and wild-type mtDNA) as previously described (11). Primers that specifically amplify wild-type or ΔmtDNA are listed in Table A. Standard curves for each qPCR primer set were generated using purified plasmids individually containing approximately 1 kb of the mtDNA fragments specific for each primer set. A Student's t-test was employed to determine the level of statistical significance.

Chromatin Immunoprecipitation (ChIP)

ChIP assays for ATFS-1 and LONP-1^{FLAG} were performed as previously described (15). Synchronized worms were cultured in liquid and harvested at early L4 stage by sucrose flotation. The worms were lysed via Teflon homogenizer in cold PBS with protease inhibitors (Roche). Cross-linking of DNA and protein was performed by treating the worms with 1.85% formaldehyde with protease inhibitors for 15 min. Glycine was added to a final concentration of 125 mM and incubated for 5 min at room temperature to quench the formaldehyde. The pellets were resuspended twice in cold PBS with protease inhibitors. Samples were sonicated in a Bioruptor (Diagenode) for 15 min at 4° C. on high intensity (30 s on and 30 s off). Samples were transferred to microfuge tubes and spun at 15,000*g for 15 min at 4° C. The supernatant was precleaned with pre-blocked ChIP-grade Pierce™ magnetic protein A/G beads (Thermo Scientific) and then incubated with Monoclonal ANTI-FLAG® M2 antibody (Sigma, F1804) or Mouse mAb IgG1 Isotype Control (Cell Signaling Technology, G3A1) rotating overnight at 4° C. The antibody-DNA complex was precipitated with protein A/G magnetic beads or protein A sepharose beads (Invitrogen). After washing, the crosslinks were reversed by incubation at 65° C. overnight. The samples were then treated with RNaseA at 37° C. for 1.5 hour followed by proteinase K at 55° C. for 2 hours. Lastly, the immunoprecipitated and input DNA were purified with ChIP DNA Clean & Concentrator (Zymo Research, D5205) and used as templates for qPCR or next generation sequencing.

mtDNA-Immunoprecipitation (mtDNA-IP) and mtDNA Quantification

mtDNA-immunoprecipitation assays were performed similarly to the previously described ATFS-1 ChIP assay described (15), however the lysates were not sonicated so that wild-type and ΔmtDNA could be quantified by qPCR. Synchronized worms were cultured in liquid and harvested at early L4 stage by sucrose flotation. The worms were lysed via Teflon homogenizer in cold PBS with protease inhibitors (Roche). Cross-linking of DNA and protein was performed by treating the worms with 1.85% formaldehyde along with protease inhibitors for 20 min at room temperature. Glycine was added to a final concentration of 125 mM and incubated for 5 min at room temperature to quench the formaldehyde. The pellets were washed twice in cold PBS with protease inhibitor. Samples were transferred to microfuge tubes and spun at 15,000*g for 15 min at 4° C. The supernatant was precleaned with pre-blocked ChIP-grade Pierce™ magnetic protein A/G beads (Thermo Scientific) and then incubated with the described antibodies rotating overnight at 4° C. The antibody-mtDNA complex was precipitated with protein A/G magnetic beads (Thermo Scientific) (LONP-1^{FLAG}) or protein A sepharose beads (Invitrogen) (for ATFS-1, POLG, TFAM or LONP-1 antibodies. Sonicated salmon sperm DNA was used to block non-specific DNA binding on beads). After washing, the crosslinks were reversed by incubation at 65° C. overnight. The samples were then treated with RNaseA at 37° C. for 1.5 hour and then proteinase K at 55° C. for 2 hours. Lastly, the samples were purified with ChIP DNA Clean & Concentrator (Zymo Research, D5205) and used as templates for qPCR. The results were normalized by input and either non-specific rabbit IgG or mouse IgG1 was used as a negative control. Primers that amplify wild-type, ΔmtDNA, or all mtDNAs (Total mtDNAs) are in Table A (11).

ChIP-Seq Analysis

The DNA fragments were sequenced using MiSeq at the University of Massachusetts Medical School Deep Sequencing Core. The quality of the raw sequencing data was first evaluated with fastqc (0.11.5). (bioinformatics.babraham.ac.uk/projects/fastqc/), and then mapped to the C. elegans genome (ce10 from UC Santa Cruz) by Burrows-Wheeler Aligner (BWA MEM, BWA version 0.7.15) algorithm with the standard default settings (35). Duplicate reads were removed using picard tools v1.96 (broadinstitute.github.io/picard/). Peaks were determined using MACS version 2.1 (36) with the no-model parameter. Input was used as a control for peak-calling. Both narrow Peaks and broad Peaks were called, and the bigwig files were generated with the signal as fold enrichment by macs2 following the procedure at github.com/taoliu/MACS/wiki/Build-Signal-Track. The final set of peaks was determined if the difference in intensity values of control sample and input had a significance level of p-value <0.01. IGV (37) was used to view the peaks and signals. To identify candidate LONP-1 interacting motifs, the regions that were highly enriched were used as input for MEME (meme.sdsc.edu). MEME was run using the parameters minw=8, maxw=25, in two modes (zoops & anr) and the significant motifs (E-value>=1e-01). A background model is used by MEME to calculate the log likelihood ratio and statistical significance of the motif.

Target Site SNP Frequency Analysis in CoxI C6930A Cells by Deep Sequencing

Library construction for deep sequencing was modified from our previous report (7). Following CDDO treatment, cells were harvested at different time points and genomic DNA extracted. Briefly, regions flanking the CoxI C6930A site were PCR amplified using locus-specific primers bearing tails complementary to the Truseq adapters as described previously (38). 50-100 ng input genomic DNA (mtDNA included) was PCR amplified with Phusion High Fidelity DNA Polymerase (New England Biolabs):(98° C., 15 s; 67° C. 25 s; 72° C. 18 s)×30 cycles. 1 μl of each PCR reaction was amplified with barcoded primers to reconstitute the TruSeq adaptors using the Phusion High Fidelity DNA Polymerase (New England Biolabs): (98° C., 15 s; 61° C., 25 s; 72° C., 18 s)×9 cycles. Equal amounts of the products were pooled and gel purified. The purified library was deep sequenced using a paired-end 150 bp Illumina MiSeq run.

MiSeq data analysis for editing at target sites or off-target sites was performed using a suite of Unix-based-software tools. First, the quality of the paired-end sequencing reads (R1 and R2 fastq files) was assessed using FastQC (bioinformatics.babraham.ac.uk/projects/fastqc/). Raw paired-end reads were combined using paired end read merger (PEAR) (39) to generate single merged high-quality full-length reads. Reads were then filtered by quality (using Filter FASTQC (38)) to remove those with a mean PHRED quality score under 30 and a minimum per base score under 24. Each group of reads was then aligned to a corresponding reference sequence using BWA (version 0.7.5) and SAMtools (version 0.1.19). To determine background SNP or sequencing errors, all reads from each negative control replicate were combined and aligned, as described above. Background SNP types and frequencies were then cataloged in a text output format at each base using bam-readcount (github.com/genome/bam-readcount). For each drug treatment group, the average background SNP frequencies (based on SNP type, position and frequency) of the triplicate negative control group were subtracted to obtain the accurate SNP frequencies.

RNA Isolation and qRT-PCR

Total RNA was isolated from worm pellets using the TRIzol™ Reagent (Invitrogen). cDNA was then synthesized from total RNA using the iScript cDNA Synthesis Kit (Bio-Rad). qPCR was performed to determine the expression levels of the indicated genes using iQ™ SYBR GREEN supermix (Bio-Rad). Primer sequences are listed in Table A. Relative expression of target genes was normalized to the control. Fold changes in gene expression were calculated using the comparative CtΔΔCt method as previously described (15). A Student's t-test was employed to determine the level of statistical significance.

Chemicals and Antibodies

CDDO (Cayman Chemicals Cat No 81035). ATFS-1 polyclonal antibodies were generated and validated previously (14). Polyclonal antibodies were generated to amino acid amino acids 1054-1072 of C. elegans POLG and subsequently affinity purified by Thermo Fisher Scientific Inc. Polyclonal antibodies were generated to amino acid amino acids 191-204 of C. elegans HMG-5 (TFAM) and subsequently affinity purified by Thermo Fisher Scientific Inc. Polyclonal antibodies were generated to amino acid amino acids 953-971 of C. elegans LONP-1 and subsequently affinity purified by Thermo Fisher Scientific Inc. Monoclonal anti-FLAG® M2 antibody (Sigma, Cat #F1804), α-tubulin (Sigma), NDUFS3 (NUO-2 in C. elegans, complex I, Abcam).

Cell Culture

The KSS cell line was a gift from Carlos Moraes (29, 30). The CoxI C6930A cell line was a gift from Giovanni Manfredi (28). Cells were cultured in DMEM (4 mM L-glutamine, 4.5 g/L glucose; Gibco, Thermo Fisher Scientific) plus 10% FBS with 1% pen-strep. Total cellular mtDNA was prepared as described (40). Cells were incubated continuously in the described concentration of CDDO for the indicated number of days. The cells were sub-cultured prior to confluence every 48 hours.

Cell Viability

At the indicated time points, cells were stained with trypan blue (41) and quantified with an automated cell counter TC-20™ (Bio-Rad). The results are an average of three independent assays.

siRNA

Cells were grown on 6-well plates and siRNAs were transfected with Lipofectamine RNAiMAX (Thermo Fisher Scientific) following the manufacturer's instructions. Human LONP1 RNAi was used in knockdown experiment (Dharmacon L-003979-00-0005) (e.g., a pool comprising GGAAAUCGGCCUACAAGAU (SEQ ID NO:1), GCGCAUGUAUGACGUGACA (SEQ ID NO:2), CGAGAGUACUUCCGCUUCA (SEQ ID NO:3), and GAAGAGACCAAUAUUCCUA (SEQ ID NO:4)).

Respiration Assays

For mitochondrial respiration assays, oxygen consumption rate (OCR) was measured using a Seahorse Extracellular Flux Analyzer XFe96 (Seahorse Biosciences) as described (40). 14,000 cells were seeded per well and OCR was measured using the Cell MitoStress Kit (as described by the manufacture). 180 μl of XF-Media was added to each well and then the plates were subjected to analysis following sequential introduction of 1.5 μM oligomycin, 1.5 μM FCCP and 0.5 μM rotenone/antimycin as indicated. Data is normalized to total protein as determined by the BCA protein assay.

Western Blots and Mitochondrial Fractionation

As previously described (42).

Imaging

Whole worm images were obtained using either a Zeiss AxioCam MRm camera mounted on a Zeiss Imager Z2 microscope or a Zeiss M2BIO dissecting scope as described (1). TMRE staining was performed by synchronizing and raising worms on plates previously soaked with S-Basal buffer containing DMSO, or final concentration 100 μM TMRE (Sigma, Cat No 87917). Prior to imaging, the TMRE-stained worms were transferred to plates seeded with control(RNAi) bacteria for 3 h to remove TMRE-containing bacteria from the digestive tract. Images were acquired using identical exposure times with a ZEISS LSM800 microscope with Airyscan. TMRE staining analysis is performed as described (43). In short, the average pixel intensity values were calculated by sampling images of different worms. The average pixel intensity for each animal was calculated using ImageJ (sb.info.nih.gov/ij/). Statistical analysis was performed using the Prism software package (GraphPad Software).

Virus Package and Transfection

ATF5 shRNA knockdown lentiviral constructs were generated using psp-108 vector (Addgene), and viruses were produced by co-transfection along with plasmids pMD2.G (Addgene) and psPAX2 (Addgene) into HEK293T cells. All viral titers were predetermined, and same number of viral particles was used for experimental and control samples. KSS cells were infected with lentiviruses, selected with puromycin for 5-7 days, and re-plated for proliferation. ATFS knockdown targeting sequences are: shRNA-1, 5′-GCTGGAACAGATGGAAGACTT-3′ (SEQ ID NO:5); shRNA-2, 5′-TGGCTCGTAGACTATGGGAAA-3′ (SEQ ID NO:6); shRNA-3, 5′-GCGCGAGATCCAGTACGTCAA-3′ (SEQ ID NO:7); see horizondiscovery.com/en/products/gene-modulation/knockdown-reagents/shrna/PIFs/TRC-Lentiviral-shRNA?nodeid=entrezgene-22809#specifications.

Statistics

All experiments were performed three times yielding similar results and comprised of biological replicates. The sample size and statistical tests were chosen based on previous studies with similar methodologies and the data met the assumptions for each statistical test performed. No statistical method was used in deciding sample sizes. In general, randomization was not used. However, the deep sequencing to quantify heteroplasmy of the CoxI G6930A mtDNA following exposure to CDDO was double-blinded. Significance was accepted at p<0.05. The researchers involved in experiments were not completely blinded during sample obtainment or data analysis. All data are reported as mean±SEM.

TABLE A
Primers for qPCR, qRT-PCR, mtDNA quantitation and gene editing

| Primer Name: | Primer sequence | SEQ ID NO: |
|---|---|---|
| C. elegans | | |
| primers | | |
| ΔmtDNA; uaDf5 | F: 5′-TTGCTTTTTCTTTATATGTTTTG-3′ | 8 |
| | R: 5′-TTTATTTAATTTGGTTAAACAAGAGGT-3′ | 9 |
| WT mtDNA | F: 5′-GCTTTTTCTTTATATGTTTTGTG-3′ | 10 |
| | R: 5′-TCACCTTCAGAAAAATCAAATGG-3′ | 11 |
| Total mtDNA | F: 5′-GCAGTCTTAGCGTGAGGACATTA-3′ | 12 |
| | R: 5′-AAACATAAAACATAAATAGAACTAACCA-3′ | 13 |
| hsp-6; qRT-PCR | F: 5′-GAAGATACGAAGACCCAGAGGTTC-3′ | 14 |
| | R: 5′-CAACCTGAGATGGGGAATACACT-3′ | 15 |
| act-3; qRT-PCR | F: 5′-ATCCGTAAGGACTTGTACGCCAAC-3′ | 16 |
| | R: 5′-CGATGATCTTGATCTTCATGGTTG-3′ | 17 |
| Cybrid cell | | |
| primers; human | | |
| KSS cells, | F: 5′-AGAGAACCAACACCTCTTTACAGTGA-3′ | 18 |
| KSS mtDNA | R: 5′-AGGGTGGGGTTATTTTCGTTAATG-3′ | 19 |
| KSS cells, | F: 5′-AAGCACTGCTTATTACAATTTTACTGGG-3′ | 20 |
| WT mtDNA | R: 5′-TTGAGCCAATAATGACGTGAAGTCC-3′ | 21 |
| Beta 2 | F: 5′-GCTGGGTAGCTCTAAACAATGTATTCA-3′ | 22 |
| myoglobin (B2M) | R: 5′-CCATGTACTAACAAATGTCTAAAATGGT-3′ | 23 |
| CoxI G6930A | 5′-ctacacgacgctcttccgatct-CCTCCGCTACCATAATCATCGCTA | 24 |
| sequencing | | |
| primer F | | |
| CoxI G6930A | 5′-agacgtgtgctcttccgatct-GTGATGAGTTTGCTAATACAATGCCAG | 25 |
| sequencing | | |
| primer R | | |
| | Lower case letters are the bar code | |
| atfs-1^{ΔNLS} | /AltR1/rArUrUrCrArArCrArUrUrCrGrArGrCrUrCrGrArUrGrUr | 26 |
| | UrUrUrArGrArGrCrUrArUrGrCrU/AltR2/ | |
| lonp1^{FLAG} | /AlTR1/rArArGrArArArGrGrGrArCrArCrGrUrArUrUrArUrGrUr | 27 |
| | UrUrUrArGrArGrCrUrArUrGrCrU/AltR2/ | |
| Replacement | | |
| templates | | |
| atfs-1^{ΔNLS} | AGAAACGTGGAGTTGTTCTGAAGCCTTCAGTCGACGAGGAAACCGATCGAGC | 28 |
| | TCGAATGTTGAATAGAATAGCAGCAGTTCGGTATAGAGAGAAGAAACGAGCC | |
| lonp-1^{FLAG} | ATATTCGATTCGTTTCGCATTATGATGAGCTCTACGAGCATCTCTTCCAAGG | 29 |
| | ATCCGACTACAAGGACGATGACGACAAGGGATCTGACTACAAGGACGATGAC | |
| | GACAAGGGATCTGACTACAAGGACGATGACGACAAGTAATAATACGTGTCCC | |
| | TTTCTTTCACCCCCAGTTAATACCTTTCAGATAGATTTTG | |

TABLE B
Antibodies

| | | Catalog | | |
|---|---|---|---|---|
| Antibody | Source | Number | Peptide sequence | SEQ ID NO: |
| HMG-5 | Thermo | Customized | LQKWEAEQKENADQ | 30 |
| (TFAM) | | | | |
| POLG | Thermo | Customized | DGVAWTVDDLLKLTGGKLD | 31 |
| LONP-1 | Thermo | Customized | ELDIRFVSHYDELYEHLFQ | 32 |
| ATFS-1 | Nargund et al., | Customized | | |
| | Science 2012 | | | |
| ANTI-FLAG ® M2 | Sigma | F1804 | | |
| Anti-α-Tubulin | Sigma | T9026 | | |
| Normal | Cell Signaling | 2729 | | |
| Rabbit IgG | Tech | | | |
| Mouse mAb | Cell Signaling | 5415 | | |
| IgG1 | Tech | | | |
| LONP1 Rabbit | Cell Signaling | 28020 | | |
| mAb | Tech | | | |
| NDUFS3 | Abcam | ab14711 | | |

### Example 1. Degradation of ATFS-1 by LONP-1 Promotes Deleterious Mitochondrial Genome Heteroplasmy

In a C. elegans model of deleterious heteroplasmy, the UPR^{mt} bZIP transcription factor ATFS-1 is required to maintain ΔmtDNAs (Lin et al., Nature 533, 416-419 (2016); Gitschlag et al., Cell Metab 24, 91-103 (2016)). UPR^{mt} inhibition caused depletion of the ΔmtDNA from ˜60% to less than 10%, but if the UPR^{mt} activation was enhanced, the ΔmtDNA accumulated at a faster rate than wild-type mtDNA suggesting the UPR^{mt} either promotes preferential replication of ΔmtDNAs (Russell et al. Sci Rep 8, 1799 (2018)) or degradation of wild-type mtDNAs (Zhou et al., Science 353, 394-399 (2016); Yu et al., Curr Biol 27, 1033-1039 (2017)).

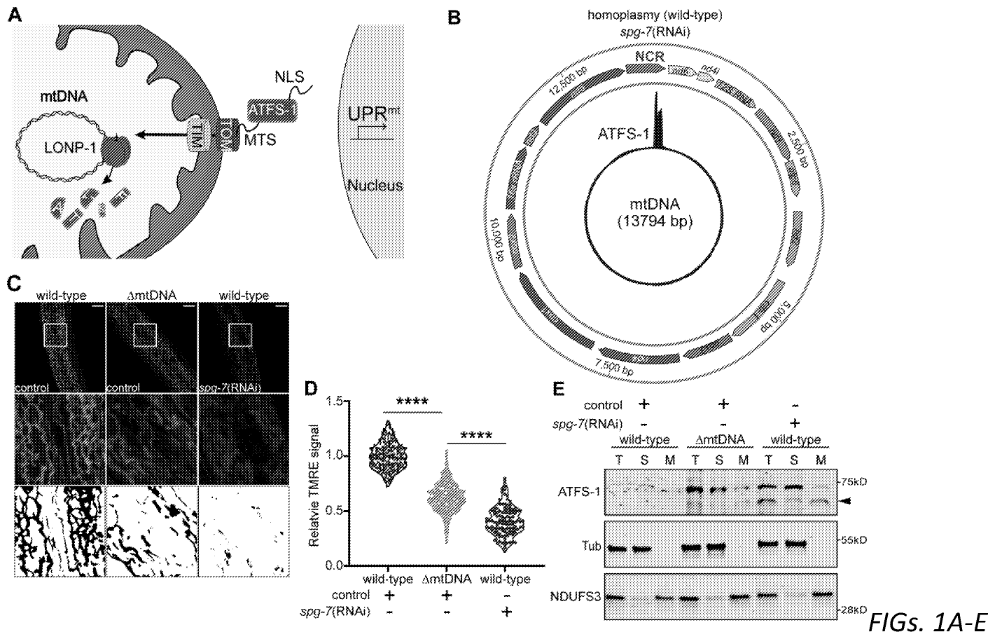

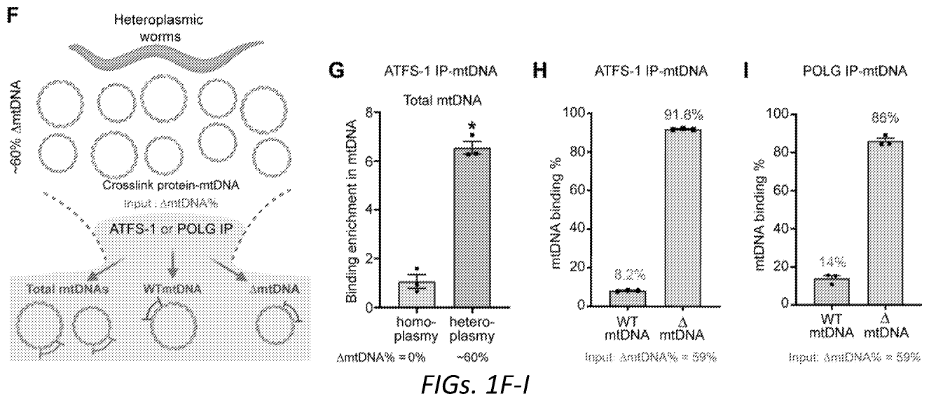

The UPR^{mt} transcription factor ATFS-1 is required to maintain ΔmtDNAs (11, 12) in a heteroplasmic C. elegans strain that harbors ˜60% ΔmtDNAs with a 3.1 kb deletion (13). ATFS-1 harbors both a nuclear localization sequence (NLS) and a mitochondrial targeting sequence (MTS) (FIG. 1A), consistent with biochemical fractionation evidence indicating that this bZIP protein resides in both compartments. In wild-type worms, the majority of ATFS-1 is imported into the mitochondrial matrix where it is degraded by the protease LONP-1 (FIG. 1A) (14, 15). However, during mitochondrial dysfunction caused by inhibition of the essential mitochondrial protease SPG-7, ATFS-1 accumulates in the nucleus (16) where it mediates a transcriptional response known as the mitochondrial unfolded protein response (UPR^{mt}) to promote survival, longevity and recovery of mitochondrial function (17). Importantly, ATFS-1 also accumulates within the mitochondrial matrix where it binds mtDNA at a single site within the regulatory non-coding region (NCR) (FIG. 1B) (15). Both mitochondrial and nuclear accumulation of ATFS-1 are required for development during mitochondrial dysfunction (11, 15). Because ATFS-1 binds mtDNA and induces over 600 mRNAs during mitochondrial dysfunction, it is unclear whether ATFS-1 promotes heteroplasmy by suppressing degradation of ΔmtDNAs or by stimulating ΔmtDNA replication.

Consistent with heteroplasmy causing mitochondrial dysfunction (18), mitochondrial membrane potential was reduced in heteroplasmic worms relative to wild-type worms (FIGS. 1C,D) although not to the extent of wild-type worms raised on spg-7(RNAi) (FIGS. 1C,D). As in spg-7(RNAi)-treated worms, ATFS-1 accumulated within mitochondria of heteroplasmic worms as determined by subcellular fractionation (FIG. 1E). As ATFS-1 binds mtDNA during mitochondrial dysfunction, we examined the interaction with mtDNA in heteroplasmic worms via IP-mtDNA followed by qPCR to quantify total, wild-type and ΔmtDNAs (FIG. 1F). While heteroplasmic worms harbored 1.25-fold more mtDNAs than wild-type worms (FIG. S1A), ATFS-1 interacted with ˜6-fold more mtDNAs in heteroplasmic worms relative to wild-type homoplasmic worms (FIG. 1G), consistent with the accumulation of ATFS-1 in dysfunctional mitochondria (FIG. 1E). Because the ATFS-1 binding site within the NCR is present in both wild-type mtDNA and ΔmtDNA, we examined binding to each genome. Strikingly, of the mtDNAs bound by ATFS-1, 91.8% were ΔmtDNAs and 8.2% were wild-type mtDNAs, which is considerably enriched relative to the ΔmtDNA ratio of 59% in whole worm lysate (FIG. 1H).

Previously, we found that inhibition of the mtDNA replicative polymerase POLG caused depletion of ΔmtDNAs in heteroplasmic worms relative to wild-type mtDNAs (11), which is similar to findings in a Drosophila model of deleterious mtDNA heteroplasmy (19). Both findings suggest increased replication of ΔmtDNAs in heteroplasmic cells. To explore the relationship between ATFS-1 and mtDNA replication, we generated POLG antibodies which detected a ˜120 KD band that co-fractionated with the OXPHOS protein NDUFS3 (FIG. S1B) and was depleted by polg(RNAi) (FIG. SIC). Interestingly, IP-mtDNA using the POLG antibody indicated that the replicative polymerase also interacted with more ΔmtDNAs than wildtype mtDNAs (FIG. 1I and FIG. S1D), similar to ATFS-1 (FIG. 1H). As a control, we generated antibodies to the mtDNA packaging protein HMG-5 (TFAM in mammals) (FIGS. S1E,F), which interacts with mtDNAs independent of replication (20). In contrast to ATFS-1 and POLG, the percentage of ΔmtDNAs bound to HMG-5 reflected the percentage within the whole worm lysate (FIG. S1G). Combined, these data indicate that ATFS-1 and a component of the replisome preferentially associate with ΔmtDNAs relative to wild-type mtDNAs in heteroplasmic worms.

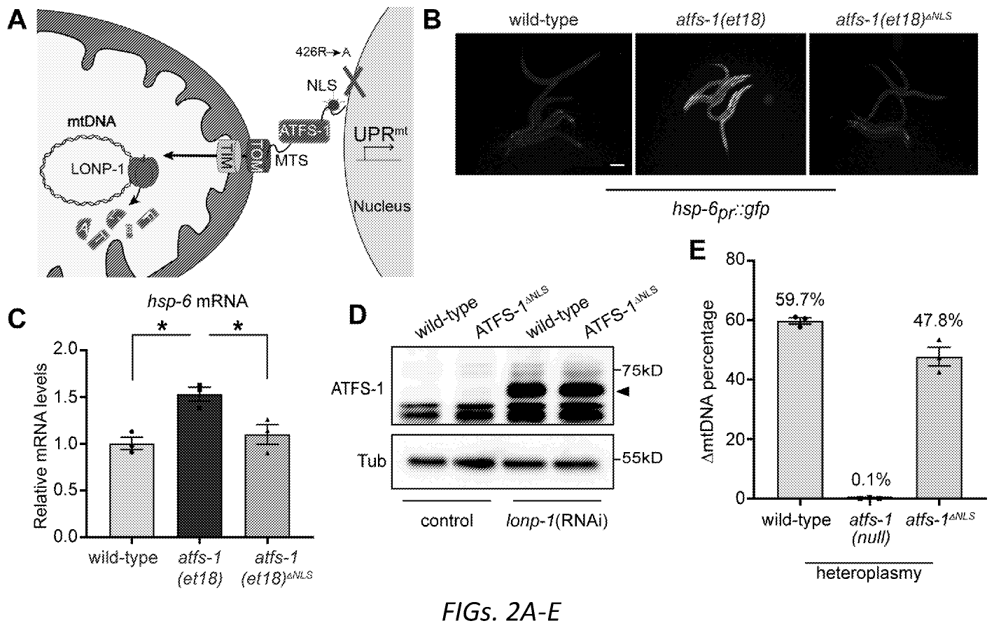

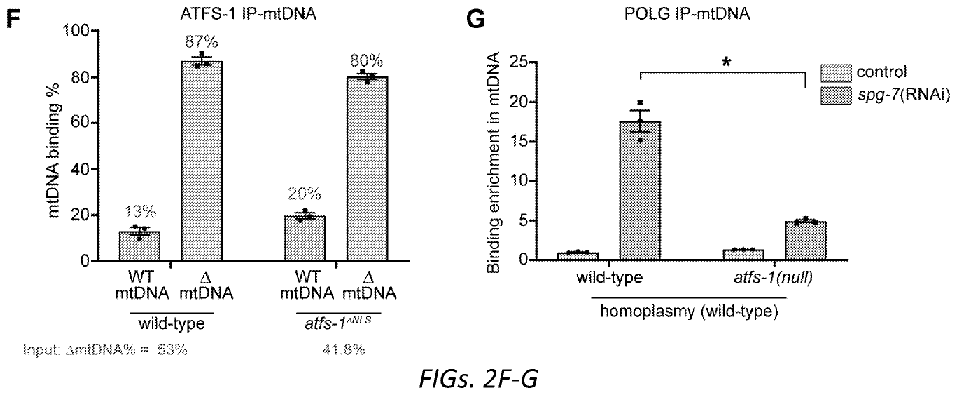

To further examine the role of mitochondrial-localized ATFS-1 in maintaining heteroplasmy, we generated a strain in which the NLS within ATFS-1 was impaired via genome editing (FIG. 2A and FIG. S2A). Consistent with atfs-1^{ΔNLS} lacking nuclear activity, induction of hsp-6 mRNA was impaired when raised on spg-7(RNAi) (FIG. S2B). The ΔNLS mutation also suppressed activation of the UPR^{mt} reporter hsp-6ₚᵣ::gfp, and hsp-6 mRNA, caused by an allele of ATFS-1 with a weak mitochondrial targeting sequence that causes constitutive UPR^{mt} activation (21) (FIGS. 2B,C). Importantly, ATFS-1's accumulated within mitochondria to similar levels as wild-type ATFS-1 upon LONP-1 inhibition (FIG. 2D) indicating the protein was expressed and processed similarly to wild-type ATFS-1. We next introduced the atfs-1(null) (22) or atfs-1^{ΔNLS} alleles into heteroplasmic worms. As expected, atfs-1(null) worms were unable to harbor any ΔmtDNAs (FIG. 2E). However, despite the lack of the atfs-1-mediated nuclear transcription program, atfs-1^{ΔNLS} worms were able to maintain ΔmtDNAs, albeit to a somewhat lesser degree than wild-type atfs-1 worms (FIG. 2E). Like ATFS-1, ATFS-1^{ΔNLS} also bound a higher percentage of ΔmtDNAs, consistent with mitochondrial-localized ATFS-1 being sufficient to maintain heteroplasmy (FIGS. 2E,F). Combined, these findings emphasize the importance of mitochondrial-localized ATFS-1 in maintaining deleterious mtDNA heteroplasmy.

We next sought to determine if mitochondrial-localized ATFS-1 is required for POLG binding to mtDNA in dysfunctional mitochondria. Because atfs-1-deletion caused complete loss of ΔmtDNAs in heteroplasmic worms (FIG. 2E), we examined the dependence of the POLG-mtDNA interaction on ATFS-1 in homoplasmic worms raised on control or spg-7(RNAi) (FIG. 2G). Similar to ATFS-1 (FIG. 1B), POLG interacted with more mtDNAs during exposure to spg-7(RNAi) (FIG. 2G). However, POLG binding to mtDNA was reduced in atfs-1(null) worms (FIG. 2G), suggesting ATFS-1 promotes POLG binding to mtDNAs in dysfunctional mitochondria. To circumvent the potentially confounding effects of the induction of polg mRNA by ATFS-1 during stress (11, 16), we performed a similar experiment in atfs-1A worms that lack nuclear activity. Like wild-type ATFS-1, ATFS-1^{ΔNLS} also promoted POLG binding to mtDNA during spg-7(RNAi) exposure, in contrast to atfs-1(null) worms (FIG. S2C). Combined, our data suggest that ATFS-1 accumulation within dysfunctional mitochondria promotes POLG recruitment to mtDNA.

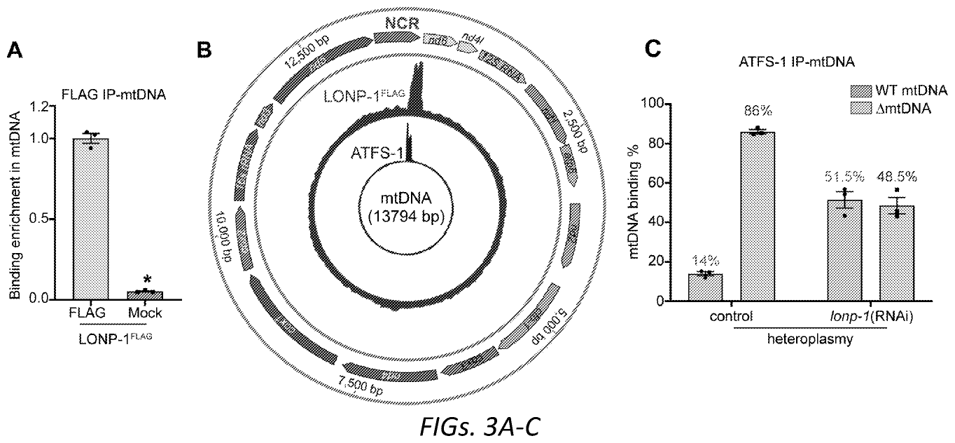

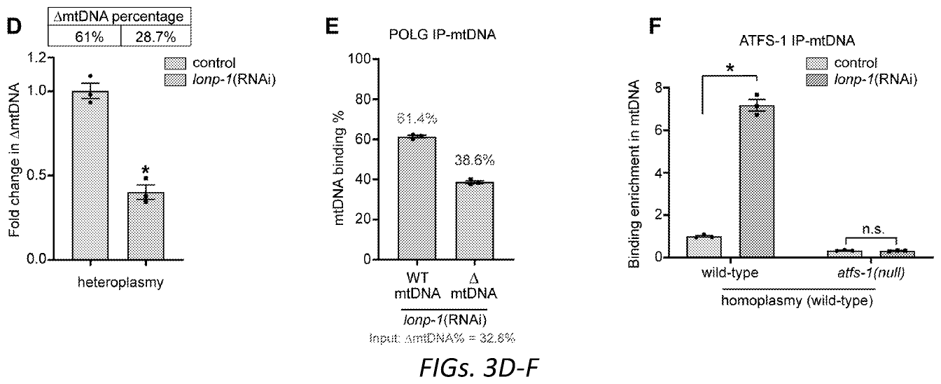

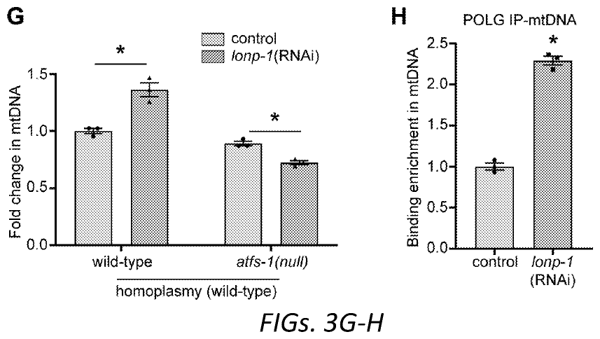

To gain insight into how the enriched association between ATFS-1 and ΔmtDNAs is established in heteroplasmic worms, we focused on the mitochondrial protease LONP-1, which is required for the degradation of ATFS-1 within the mitochondrial matrix of wild-type worms (FIGS. 1A and 2D) (16). LONP-1 is known to interact with mtDNA in diverse species (23, 24), and has been shown to regulate mtDNA replication (25, 26), suggesting a role in regulating heteroplasmy. We generated antibodies to C. elegans LONP-1 (FIG. S3A), as well as a strain in which the C-terminus of LONP-1 was epitope tagged via genome editing (FIGS. S3B,C,D,E). As expected, LONP-1 interacted with mtDNA in C. elegans (FIG. 3A, S4A) And, unlike ATFS-1, LONP-1 interacted similarly with wildtype and ΔmtDNAs (FIG. S4B) suggesting LONP-1 is constitutively bound while ATFS-1 binding is likely transient. Lastly, we sought to determine where in the mtDNA LONP-1^{FLAG} binds. LONP-1^{FLAG} ChIP-seq indicated that LONP-1 bound several G-rich sites throughout mtDNA (FIG. S4C), but was especially enriched within the NCR (FIG. 3B). Interestingly, the strongest LONP-1^{FLAG} peak within the NCR overlapped with the ATFS-1 binding site (FIG. 3B and FIG. S4D) suggesting it may regulate ATFS-1 binding to mtDNA.

We next examined the interaction between ATFS-1 and mtDNAs in heteroplasmic worms upon LONP-1 inhibition. As before, ATFS-1 binding was enriched on ΔmtDNAs (FIG. 3C). However, lonp-1(RNAi) exposure resulted in ATFS-1 binding nearly equally to wild-type mtDNAs and ΔmtDNAs indicating the mtDNA-bound protease is required for the enriched interaction between ATFS-1 and ΔmtDNAs (FIG. 3C). Interestingly, lonp-1 inhibition via RNAi caused a 2-fold reduction of ΔmtDNAs (FIG. 3D) and a concomitant increase in wild-type mtDNAs improving the heteroplasmy ratio from 61% ΔmtDNAs to 28.7% (FIG. 3D). Similar results were obtained in atfs-1^{ΔNLS} worms upon LONP-1 inhibition (FIG. S5A). In addition to increasing the percentage of wild-type mtDNAs bound by ATFS-1, lonp-1(RNAi) also increased the percentage of POLG that interacted with wild-type mtDNAs while reducing the amount bound to ΔmtDNAs (FIG. 3E). Importantly, lonp-1(RNAi) did not alter the percentage of HMG-5/TFAM bound to ΔmtDNAs, consistent with HMG-5 interacting with all mtDNAs (FIG. S5B). Combined, these findings support a role for LONP-1-mediated degradation of mitochondrial ATFS-1 in promoting ΔmtDNA propagation.

We next examined the impact of lonp-1(RNAi) on mtDNA accumulation in homoplasmic worms. Exposure to lonp-1(RNAi) resulted in increased ATFS-1 binding to mtDNA (FIG. 3F) and an increase in total mtDNA (FIG. 3G), suggesting the ATFS-1:mtDNA interaction promotes replication. Furthermore, lonp-1(RNAi) also caused increased POLG:mtDNA binding (FIG. 3H). Moreover, in atfs-1_{ΔNLS} worms, LONP-1 inhibition also increased total mtDNA number (FIG. S5C). However, the increase in mtDNA caused by LONP-1 inhibition was abolished in atfs-1(null) worms (FIG. 3G). Together, these findings support a role for mitochondrial-localized ATFS-1 in promoting mtDNA replication in a manner that is negatively regulated by LONP-1-dependent degradation of ATFS-1.

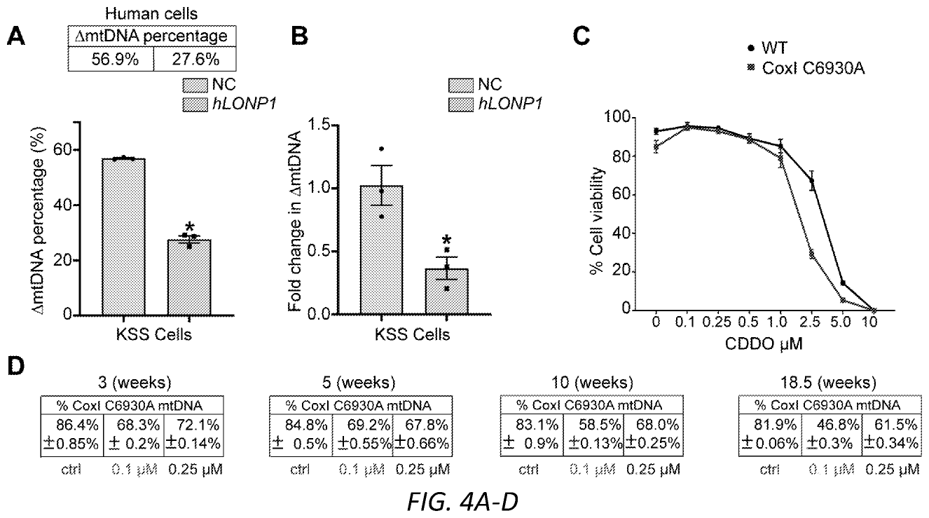

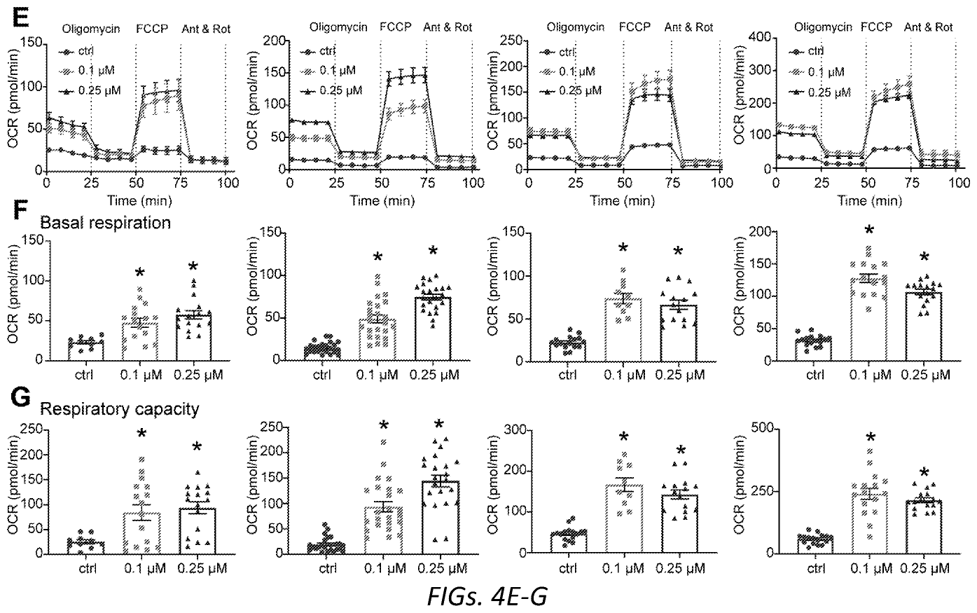

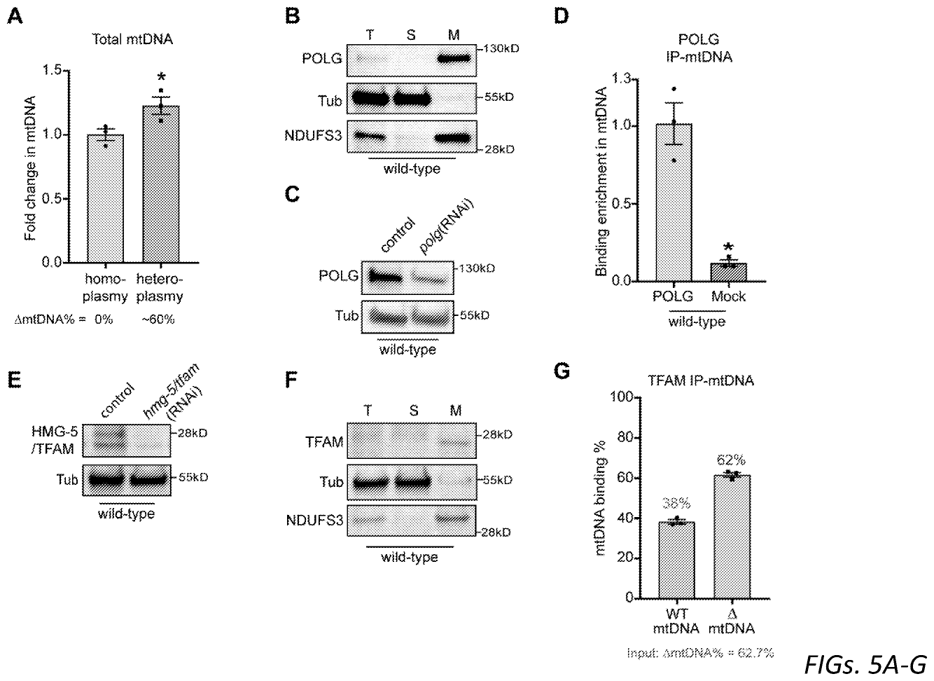

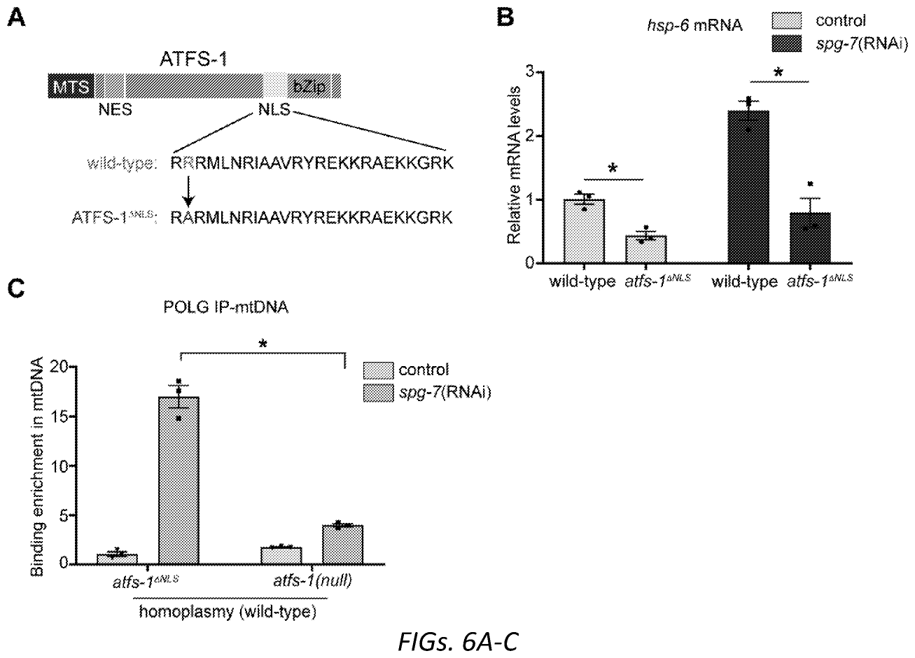

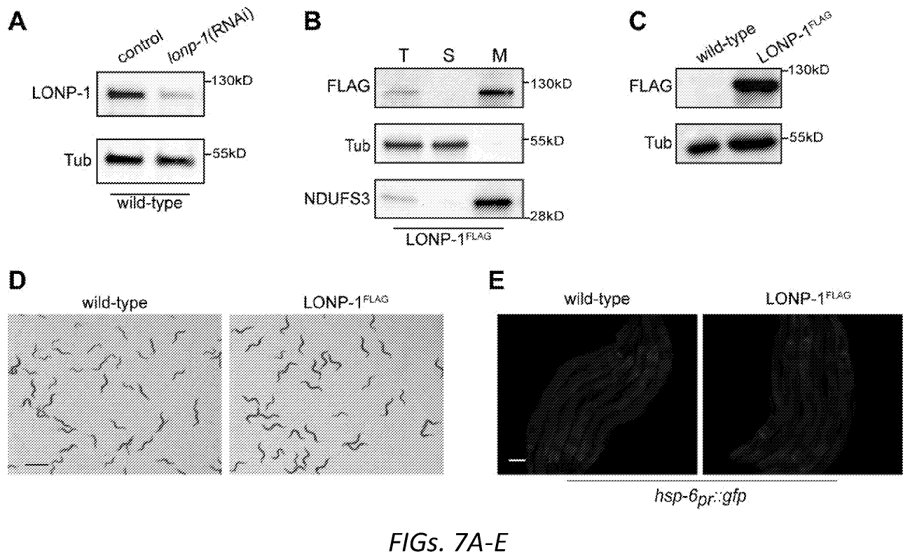

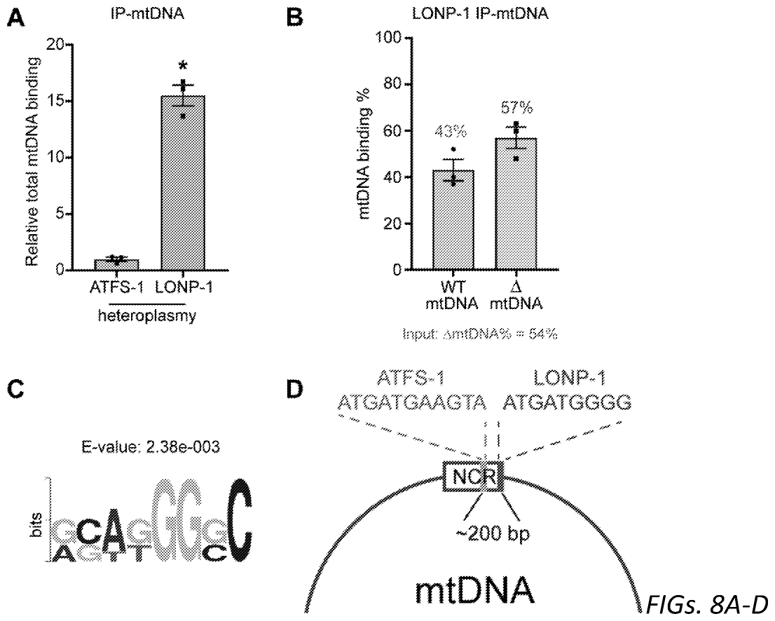

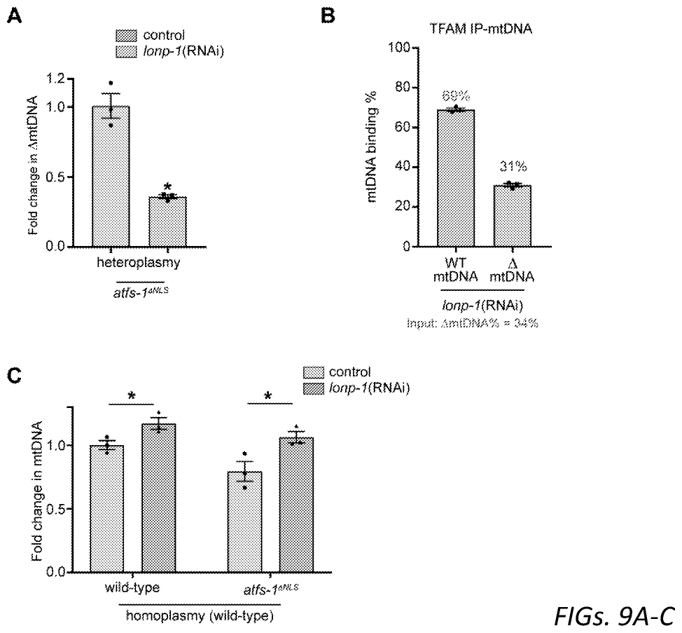

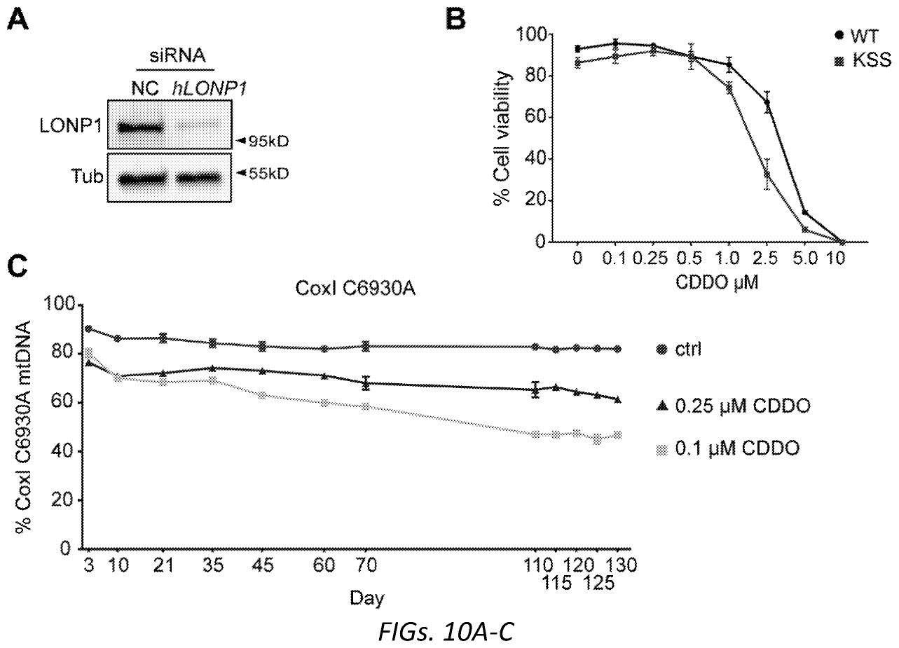

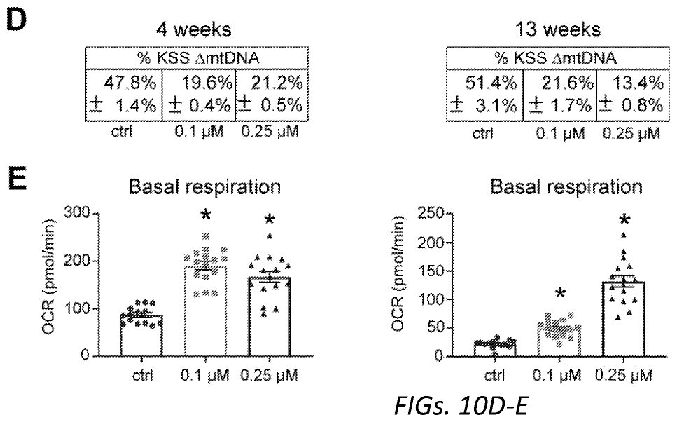

Lastly, we examined whether the role of LONP1 in maintaining ΔmtDNAs is conserved in mammals by examining two human heteroplasmic cybrid cell lines, which harbor a combination of wild-type mtDNA and ΔmtDNAs associated with mitochondrial disease (27). One cybrid line harbors a single nucleotide transition (COXI G6930A) that introduces a premature stop codon in the cytochrome c oxidase subunit I gene isolated from a patient with a multisystem mitochondrial disorder (28). We also examined a cybrid line harboring a 4977 base pair deletion known as the “common deletion” which removes multiple OXPHOS genes and is associated with Kearns-Sayre Syndrome (KSS), progressive external ophthalmoplegia, cancer and aging (29-32). We first examined the impact of LONP1 siRNA on heteroplasmy in the KSS cybrid line. Similar to inhibition of C. elegans LONP-1, inhibition of human LONP1 by siRNA for 4 days (FIG. S6A) improved the heteroplasmy ratio from 56.9% to 27.6% KSS mtDNA (FIG. 4A). Similarly, ΔmtDNAs were decreased 2.7-fold suggesting LONP1's role in promoting heteroplasmy is conserved in mammals (FIG. 4B).

To further investigate the role of LONP1 protease activity, we used the LONP1 inhibitor CDDO (2-cyano-3,12-dioxo-oleana-1,9(11)-dien-28-oic acid), also known as Bardoxolone (33). 0.1 μM and 0.25 μM CDDO were used, as neither concentration affected viability of the KSS or CoxI G6930A cell lines (FIG. 4C and FIG. S6B). As determined by deep sequencing, the CoxI G6930A cybrid line initially harbored 86% G6930A mutant mtDNA and 14% wild-type mtDNA (FIG. 4D and FIG. S6C). Impressively, incubation with 0.1 μM or 0.25 μM CDDO for 3 weeks resulted in depletion of the CoxI G6930A mtDNA from 86% to 68% and 72%, respectively, with a relative increase in wild-type mtDNAs (FIG. 4D and FIG. S6C), Furthermore, continuous incubation with 0.1 μM or 0.25 μM CDDO for 18.5 weeks further decreased the heteroplasmic ratios from ˜90% to ˜47% and 62%, respectively (FIG. 4D and FIG. S6C). Mitochondrial respiratory function was also measured at time points throughout the time course (FIGS. 4E-G). Impressively, depletion of the CoxI G6930A mtDNA resulted in increased basal respiration and maximal respiratory capacity (FIGS. 4E-G). For example, 3 weeks exposure to 0.1 μM CDDO improved basal oxygen consumption ˜2-fold (FIGS. 4E,F), while exposure for 18.5 weeks improved basal oxygen consumption over 3-fold (FIGS. 4E,F).

Similar results were obtained when the KSS cybrid cells were incubated with CDDO. As determined by qPCR, KSS cells initially harbored ˜50% ΔmtDNA. Incubation with 0.1 μM or 0.25 μM CDDO for 4 weeks depleted ΔmtDNAs to 19.6% and 21.2%, respectively, alongside a concomitant ˜2-fold increase in basal oxygen consumption (FIGS. S6D,E). These findings suggest that LONP1 promotes propagation of deleterious mtDNAs independent of genome length, as maintenance of mutant mtDNAs with either a large deletion or a single base pair substitution require LONP1 function.

In summary, these results demonstrate that degradation of mitochondrial-localized ATFS-1 by the protease LONP-1 regulates the propagation of deleterious mtDNAs. We propose that under basal conditions, this mechanism enables compartment-autonomous regulation of mtDNA replication that responds to functional heterogeneity within the mitochondrial network. In such a model, a decline in mitochondrial function would be accompanied by impaired LONP1 activity, resulting in accumulation of ATFS-1 within mitochondria, increased binding of ATFS-1 to mtDNA, and recruitment of the replisome to mtDNA. However, if compartmental dysfunction arises because of ΔmtDNAs, these deleterious genomes are preferentially replicated. Inhibiting LONP1 throughout the mitochondrial network may negate this preferential replication, leading to a reduction in the heteroplasmic ratio and recovery of mitochondrial function.

### Example 2. ATF5 shRNA Improves Heteroplasmy in Human Cells

The human homolog of worm ATFS-1 is ATF5. We previously found that ATF5 expression in worms lacking functional ATFS-1, was able to rescue transcription of the mitochondrial chaperone hsp-60 during mitochondrial dysfunction (Fiorese et al., Curr Biol. 2016 Aug. 8; 26(15):2037-2043). Furthermore, in cultured human cells ATF5 regulates the transcription of similar genes to that of ATFS-1 including the mitochondrial chaperones HSP60 and mtHSP70, as well as the mitochondrial protease LONP-1.

ATF5 shRNA improves heteroplasmy in human cells (see FIGS. 11A-E), which is similar to the findings in C. elegans where ATFS-1 inhibition also improves heteroplasmy. In humans, the synthesis of ATF5 protein requires the phosphorylation of the translation initiation factor eIF2α (Zhou et al., J Biol Chem. 2008 Mar. 14; 283(11):7064-73). The SSRI (Selective Serotonin Reuptake Inhibitor) Trazadone has been shown to inhibit the outputs of eIF2α phosphorylation (Halliday et al., Brain. 2017 Jun. 1; 140(6):1768-1783), which included synthesis of ATF5 and LONP1, through a mechanism unrelated to its SSRI activity (Id). Similar to ATF5 shRNA, multiple concentrations of Trazadone also improved heteroplasmy (see FIG. 11E).

### REFERENCES

1. S. Srivastava, C. T. Moraes, Manipulating mitochondrial DNA heteroplasmy by a mitochondrially targeted restriction endonuclease. Human molecular genetics 10, 3093-3099 (2001).
2. M. Tanaka et al., Gene therapy for mitochondrial disease by delivering restriction endonucleaseSmaI into mitochondria. Journal of biomedical science 9, 534-541 (2002).
3. D. F. Suen, D. P. Narendra, A. Tanaka, G. Manfredi, R. J. Youle, Parkin overexpression selects against a deleterious mtDNA mutation in heteroplasmic cybrid cells. Proceedings of the National Academy of Sciences of the United States of America 107, 11835-11840 (2010).
4. Y. Zhang et al., PINK1 inhibits local protein synthesis to limit transmission of deleterious mitochondrial DNA mutations. Molecular cell 73, 1127-1137. e1125 (2019).
5. A. Hahn, S. Zuryn, The Cellular Mitochondrial Genome Landscape in Disease. Trends Cell Biol 29, 227-240 (2019).
6. C. V. Pereira, C. T. Moraes, Current strategies towards therapeutic manipulation of mtDNA heteroplasmy. Front Biosci (Landmark Ed) 22, 991-1010 (2017).
7. N. G. Larsson, D. A. Clayton, Molecular genetic aspects of human mitochondrial disorders. Annu Rev Genet 29, 151-178 (1995).
8. E. A. Schon, S. DiMauro, M. Hirano, R. W. Gilkerson, Therapeutic prospects for mitochondrial disease. Trends Mol Med 16, 268-276 (2010).
9. L. C. Greaves et al., Clonal expansion of early to mid-life mitochondrial DNA point mutations drives mitochondrial dysfunction during human ageing. PLoS genetics 10, e1004620 (2014).
10. A. Bender et al., High levels of mitochondrial DNA deletions in substantia nigra neurons in aging and Parkinson disease. Nature genetics 38, 515-517 (2006).
11. Y. F. Lin et al., Maintenance and propagation of a deleterious mitochondrial genome by the mitochondrial unfolded protein response. Nature 533, 416-419 (2016).
12. B. L. Gitschlag et al., Homeostatic Responses Regulate Selfish Mitochondrial Genome Dynamics in C. elegans. Cell Metab 24, 91-103 (2016).
13. W. Y. Tsang, B. D. Lemire, Stable heteroplasmy but differential inheritance of a large mitochondrial DNA deletion in nematodes. Biochem Cell Biol 80, 645-654 (2002).
14. A. M. Nargund, M. W. Pellegrino, C. J. Fiorese, B. M. Baker, C. M. Haynes, Mitochondrial import efficiency of ATFS-1 regulates mitochondrial UPR activation. Science 337, 587-590 (2012).
15. A. M. Nargund, C. J. Fiorese, M. W. Pellegrino, P. Deng, C. M. Haynes, Mitochondrial and nuclear accumulation of the transcription factor ATFS-1 promotes OXPHOS recovery during the UPR(mt). Molecular cell 58, 123-133 (2015).
16. A. M. Nargund, M. W. Pellegrino, C. J. Fiorese, B. M. Baker, C. M. Haynes, Mitochondrial import efficiency of ATFS-1 regulates mitochondrial UPR activation. Science 337, 587-590 (2012).
17. T. Shpilka, C. M. Haynes, The mitochondrial UPR: mechanisms, physiological functions and implications in ageing. Nature reviews. Molecular cell biology 19, 109-120 (2018).
18. Y. F. Lin et al., Maintenance and propagation of a deleterious mitochondrial genome by the mitochondrial unfolded protein response. Nature, (2016).
19. A. C.-Y. Chiang, E. McCartney, P. H. O'Farrell, H. Ma, A genome-wide screen reveals that reducing mitochondrial DNA polymerase can promote elimination of deleterious mitochondrial mutations. Current Biology 29, 4330-4336. e4333 (2019).
20. C. Kukat et al., Cross-strand binding of TFAM to a single mtDNA molecule forms the mitochondrial nucleoid. Proc Natl Acad Sci USA 112, 11288-11293 (2015).
21. M. Rauthan, P. Ranji, N. Aguilera Pradenas, C. Pitot, M. Pilon, The mitochondrial unfolded protein response activator ATFS-1 protects cells from inhibition of the mevalonate pathway. Proceedings of the National Academy of Sciences of the United States of America 110, 5981-5986 (2013).
22. P. Deng et al., Mitochondrial UPR repression during Pseudomonas aeruginosa infection requires the bZIP protein ZIP-3. Proceedings of the National Academy of Sciences of the United States of America, 116(13):6146-6151 (2019).
23. T. Liu et al., DNA and RNA binding by the mitochondrial lon protease is regulated by nucleotide and protein substrate. The Journal of biological chemistry 279, 13902-13910 (2004).
24. S. H. Chen, C. K. Suzuki, S. H. Wu, Thermodynamic characterization of specific interactions between the human Lon protease and G-quartet DNA. Nucleic acids research 36, 1273-1287 (2008).
25. Y. Matsushima, Y. I. Goto, L. S. Kaguni, Mitochondrial Lon protease regulates mitochondrial DNA copy number and transcription by selective degradation of mitochondrial transcription factor A (TFAM). Proceedings of the National Academy of Sciences 107, 18410-18415 (2010).
26. A. Goke et al., Mrx6 regulates mitochondrial DNA copy number in S. cerevisiae by engaging the evolutionarily conserved Lon protease Pim1. Molecular Biology of the Cell, mbc.E19-08-0470 (2019).
27. M. P. King, G. Attardi, Human cells lacking mtDNA: repopulation with exogenous mitochondria by complementation. Science 246, 500-503 (1989).
28. C. Bruno et al., A stop-codon mutation in the human mtDNA cytochrome c oxidase I gene disrupts the functional structure of complex IV. Am J Hum Genet 65, 611-620 (1999).
29. C. T. Moraes et al., Mitochondrial DNA deletions in progressive external ophthalmoplegia and Kearns-Sayre syndrome. N Engl J Med 320, 1293-1299 (1989).
30. C. T. Moraes, E. A. Schon, S. DiMauro, A. F. Miranda, Heteroplasmy of mitochondrial genomes in clonal cultures from patients with Kearns-Sayre syndrome. Biochem Biophys Res Commun 160, 765-771 (1989).
31. A. A. M. Yusoff W. S. W. Abdullah, S. Khair, S. M. A. Radzak, A comprehensive overview of mitochondrial DNA 4977-bp deletion in cancer studies. Oncol Rev 13, 409 (2019).
32. H. C. Lee, C. Y. Pang, H. S. Hsu, Y. H. Wei, Differential accumulations of 4,977 bp deletion in mitochondrial DNA of various tissues in human ageing. Biochim Biophys Acta 1226, 37-43 (1994).
33. S. H. Bernstein et al., The mitochondrial ATP-dependent Lon protease: a novel target in lymphoma death mediated by the synthetic triterpenoid CDDO and its derivatives. Blood 119, 3321-3329 (2012).
34. A. Paix, A. Folkmann, D. Rasoloson, G. Seydoux, High Efficiency, Homology-Directed Genome Editing in Caenorhabditis elegans Using CRISPR-Cas9 Ribonucleoprotein Complexes. Genetics 201, 47-54 (2015).
35. H. Li, R. Durbin, Fast and accurate short read alignment with Burrows-Wheeler transform. Bioinformatics 25, 1754-1760 (2009).
36. Y. Zhang et al., Model-based analysis of ChIP-Seq (MACS). Genome Biol 9, R137 (2008).
37. J. T. Robinson et al., Integrative genomics viewer. Nat Biotechnol 29, 24-26 (2011).
38. D. Blankenberg et al., Manipulation of FASTQ data with Galaxy. Bioinformatics 26, 1783-1785 (2010).
39. J. Zhang, K. Kobert, T. Flouri, A. Stamatakis, PEAR: a fast and accurate Illumina Paired-End reAd mergeR. Bioinformatics 30, 614-620 (2014).
40. C. J. Fiorese et al., The Transcription Factor ATF5 Mediates a Mammalian Mitochondrial UPR. Curr Biol 26, 2037-2043 (2016).
41. T.-N. D. Pham, W. Ma, D. Miller, L. Kazakova, S. Benchimol, Erythropoietin inhibits chemotherapy-induced cell death and promotes a senescence-like state in leukemia cells. Cell death & disease 10, 22 (2019).
42. C. M. Haynes, Y. Yang, S. P. Blais, T. A. Neubert, D. Ron, The matrix peptide exporter HAF-1 signals a mitochondrial UPR by activating the transcription factor ZC376.7 in C. elegans. Mol Cell 37, 529-540 (2010).
43. K. Palikaras, E. Lionaki, N. Tavernarakis, Coordination of mitophagy and mitochondrial biogenesis during ageing in C. elegans. Nature 521, 525-528 (2015).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for depleting deleterious mitochondrial genomes (ΔmtDNAs) in a cell, the method comprising administering an effective amount of an inhibitor of LONP1.

2. The method of claim 1, wherein administering the inhibitor results in a compensatory increase in wild type (WT) mtDNAs.

3. The method of claim 1, wherein the inhibitor of LONP1 is an inhibitory nucleic acid targeting LONP1 or ATF5.

4. The method of claim 3, wherein the inhibitory nucleic acid targeting LONP1 is an antisense oligonucleotide, single- or double-stranded RNA interference (RNAi) compound.

5. The method of claim 3, wherein the inhibitory nucleic acid targeting LONP1 is or comprises a locked nucleic acid (LNA) or peptide nucleic acid (PNA).

6. The method of claim 1, wherein the inhibitor of LONP1 is a small molecule inhibitor.

7. The method of claim 6, wherein the small molecule inhibitor of LONP1 is an oleanane triterpenoid; MG262 (Z-Leu-Leu-Leu-B(OH)₂); MG132 (carbobenzoxy-Leu-Leu-leucinal); Obtusilactone A (OA); or (-)-sesamin, or is trazadone.

8. The method of claim 7, wherein the oleanane triterpenoid is 2-cyano-3, 12-dioxooleana-1,9(11)-dien-28-oic acid (CDDO), or a derivative thereof.

9. The method of claim 8, wherein the derivative of CDDO is a methyl ester derivative (CDDO-Me) or imidazole derivative (CDDO-Im).

10. The method of claim 1, wherein the cell is in a mammalian subject.

11. The method of claim 10, wherein the cell is in a subject who has a disorder associated with ΔmtDNAs.

12. The method of claim 11, wherein the disorder is Leigh Syndrome (Subacute necrotizing encephalomyopathy); Kearns-Sayre Syndrome (KSS); Neuropathy, Ataxia and Retinitis Pigmentosa (NARP) Syndrome; Leber Hereditary Optic Neuropathy (LHON); mitochondrial encephalopathy with lactic acidosis and strokelike episodes (MELAS); Chronic Progressive External Ophthalmoplegia (CPEO); Mitochondrial Neuro-GastroIntestinal Encephalopathy (MNGIE); myoclonic epilepsy with ragged-red fibres (MERRF).

13.-24. (canceled)

25. The method of claim 10, wherein the mammalian subject is a human subject.


SEQUENCE LISTING (37 sequences)

SEQ ID NO: 1 - RNA, 19 nt, Artificial Sequence
  Human LONP1 RNAi
ggaaaucggccuacaagau

SEQ ID NO: 2 - RNA, 19 nt, Artificial Sequence
  Human LONP1 RNAi
gcgcauguaugacgugaca

SEQ ID NO: 3 - RNA, 19 nt, Artificial Sequence
  Human LONP1 RNAi
cgagaguacuuccgcuuca

SEQ ID NO: 4 - RNA, 19 nt, Artificial Sequence
  Human LONP1 RNAi
gaagagaccaauauuccua

SEQ ID NO: 5 - DNA, 21 bp, Artificial Sequence
  ATF5 targeting sequence shRNA-1
gctggaacagatggaagactt

SEQ ID NO: 6 - DNA, 21 bp, Artificial Sequence
  ATF5 targeting sequence shRNA-2
tggctcgtagactatgggaaa

SEQ ID NO: 7 - DNA, 21 bp, Artificial Sequence
  ATF5 targeting sequence shRNA-3
gcgcgagatccagtacgtcaa

SEQ ID NO: 8 - DNA, 23 bp, Artificial Sequence
  Primer C. elegans delta mtDNA; uaDf5
ttgctttttctttatatgttttg

SEQ ID NO: 9 - DNA, 27 bp, Artificial Sequence
  Primer C. elegans delta mtDNA; uaDf5
tttatttaatttggttaaacaagaggt

SEQ ID NO: 10 - DNA, 23 bp, Artificial Sequence
  primer C. elegans WT mtDNA
gctttttctttatatgttttgtg

SEQ ID NO: 11 - DNA, 23 bp, Artificial Sequence
  primer C. elegans WT mtDNA
tcaccttcagaaaaatcaaatgg

SEQ ID NO: 12 - DNA, 23 bp, Artificial Sequence
  primer C. elegans total mtDNA
gcagtcttagcgtgaggacatta

SEQ ID NO: 13 - DNA, 28 bp, Artificial Sequence
  primer C. elegans total mtDNA
aaacataaaacataaatagaactaacca

SEQ ID NO: 14 - DNA, 24 bp, Artificial Sequence
  primer C. Elegans hsp-6; qRT-PCR
gaagatacgaagacccagaggttc

SEQ ID NO: 15 - DNA, 23 bp, Artificial Sequence
  primer C. Elegans hsp-6; qRT-PCR
caacctgagatggggaatacact

SEQ ID NO: 16 - DNA, 24 bp, Artificial Sequence
  primer C. elegans act-3; qRT-PCR
atccgtaaggacttgtacgccaac

SEQ ID NO: 17 - DNA, 24 bp, Artificial Sequence
  primer C. elegans act-3; qRT-PCR
cgatgatcttgatcttcatggttg

SEQ ID NO: 18 - DNA, 26 bp, Artificial Sequence
  primer Cybrid cell primers; human KSS cells, KSS mtDNA
agagaaccaacacctctttacagtga

SEQ ID NO: 19 - DNA, 24 bp, Artificial Sequence
  primer Cybrid cell primers; human KSS cells, KSS mtDNA
agggtggggttattttcgttaatg

SEQ ID NO: 20 - DNA, 28 bp, Artificial Sequence
  primer Cybrid cell primers; human KSS cells, WT mtDNA
aagcactgcttattacaattttactggg

SEQ ID NO: 21 - DNA, 25 bp, Artificial Sequence
  primer Cybrid cell primers; human KSS cells, WT mtDNA
ttgagccaataatgacgtgaagtcc

SEQ ID NO: 22 - DNA, 27 bp, Artificial Sequence
  primer Cybrid cell primers; human Beta 2 myoglobin (B2M)
gctgggtagctctaaacaatgtattca

SEQ ID NO: 23 - DNA, 28 bp, Artificial Sequence
  primer Cybrid cell primers; human Beta 2 myoglobin (B2M)
ccatgtactaacaaatgtctaaaatggt

SEQ ID NO: 24 - DNA, 46 bp, Artificial Sequence
  primer Cybrid cell primers; human CoxI G6930A sequencing primer F
ctacacgacgctcttccgatctcctccgctaccataatcatcgcta

SEQ ID NO: 25 - DNA, 48 bp, Artificial Sequence
  primer Cybrid cell primers; human CoxI G6930A sequencing primer R
agacgtgtgctcttccgatctgtgatgagtttgctaatacaatgccag

SEQ ID NO: 26 - RNA, 36 nt, Artificial Sequence
  primer crRNAs
auucaacauucgagcucgauguuuuagagcuaugcu

SEQ ID NO: 27 - RNA, 36 nt, Artificial Sequence
  primer crRNAs
aagaaagggacacguauuauguuuuagagcuaugcu

SEQ ID NO: 28 - DNA, 104 bp, Artificial Sequence
  primer replacement templates
agaaacgtggagttgttctgaagccttcagtcgacgaggaaaccgatcgagctcgaatgttgaatagaatagcagcagttcggtatagagagaagaaacgagcc

SEQ ID NO: 29 - DNA, 196 bp, Artificial Sequence
  primer Replacement templates
atattcgattcgtttcgcattatgatgagctctacgagcatctcttccaaggatccgactacaaggacgatgacgacaagggatctgactacaaggacgatgacgacaagggatctgactacaaggacgatgacgacaagtaataatacgtgtccctttctttcacccccagttaatacctttcagatagattttg

SEQ ID NO: 30 - protein, 14 aa, Artificial Sequence
  HMG-5 (TFAM) peptide
LQKWEAEQKENADQ

SEQ ID NO: 31 - protein, 19 aa, Artificial Sequence
  POLG peptide
DGVAWTVDDLLKLTGGKLD

SEQ ID NO: 32 - protein, 19 aa, Artificial Sequence
  LONP-1 peptide
ELDIRFVSHYDELYEHLFQ

SEQ ID NO: 33 - protein, 25 aa, Artificial Sequence
  ATFS-1 wild type nuclear localization sequence
RRRMLNRIAAVRYREKKRAEKKGRK

SEQ ID NO: 34 - protein, 25 aa, Artificial Sequence
  ATFS-1deltaNLS
RARMLNRIAAVRYREKKRAEKKGRK

SEQ ID NO: 35 - DNA, 8 bp, Artificial Sequence
  LONP-1 consensus binding site within mtDNA
  misc_feature (1)..(1): n= g or a
  misc_feature (2)..(2): n= c or g
  misc_feature (3)..(3): n= a or t
  misc_feature (4)..(4): n= g or t
  misc_feature (7)..(7): n= g or c
nnnnggnc

SEQ ID NO: 36 - DNA, 11 bp, Artificial Sequence
  putative ATFS-1 binding site
atgatgaagta

SEQ ID NO: 37 - DNA, 9 bp, Artificial Sequence
  LONP-1 binding sites
atgatgggg